(19) United States
(12) Patent Application Publication
(10) Pub. No.: US 2008/0274998 A1
(43) Pub. Date: Nov. 6, 2008

(54) Disaccharide Molecules and Derivatives Thereof and Methods of Using Same

(75) Inventors: Irun R. Cohen, Rechovot (IL); Liora Cahalon, Givataim (IL); Ofer Lider, Kfar Bilu Beit (IL); Osnat Lider, Kfar Bilu Beit (IL); Amiram Ariel, Rehovot (IL)

Correspondence Address: Martin D. Moynihan;PRTSI, P.O.Box 16446, Arlington, VA, 22215, US

(73) Assignee: YEDA RESEARCH AND DEVELOPMENT CO. LTD., Rehovot (IL)

(21) Appl. No.: 10/589,754

(22) PCT Filed: Feb. 17, 2005
(86) PCT No.: PCT/IL2005/000197

§ 371 Date: Jul. 10, 2008

Related U.S. Application Data
(60) Provisional application No. 60/544,305, filed on Feb. 17, 2004.

(51) Int. Cl. A61K 31/715; A61K 31/727; A61P 35/00; A61P 31/04; A61P 31/12; A61P 29/00; A61P 37/00; C12N 5/06

(52) U.S. Cl. 514/53; 514/56; 435/375

(57) ABSTRACT

The present invention is of a method and compounds for mediating a biological activity mediated by moesin, and in particular, for such a method and compounds for mediating a biological activity that is capable of being mediated through binding of a disaccharide to moesin.

### FIELD OF THE INVENTION

The present invention relates to a method and compounds for mediating a biological activity mediated by moesin, and in particular, for such a method and compounds for mediating a biological activity that is capable of being mediated through binding of a disaccharide to moesin.

### BACKGROUND OF THE INVENTION

Moesin is a 78 kDa protein that belongs to the membrane-cytoskeleton linker proteins, it is highly homologous to radixin and ezrin and the three proteins are collectively termed ERM proteins. These proteins are localized at regions where actin is associated with the cell membrane and are thought to play a role in cell-cell adhesion, ruffling membranes and formation of microvilli. Indeed, these proteins have been shown to be associated with cell adhesion and morphogenesis. Lankes, et al., Proc. Natl. Acad. Sci. U.S.A., 88:8297 (1991); and Serrador et al., J. Cell Biol., 138:1409 (1997); Tsukita et al., J. Cell Biol. 126:391 (1994).

The proteins of the ERM family are known to function as membrane-cytoskeleton linkers, since their conserved approximately 100 amino acid C-terminal domain binds F-actin, and their conserved approximately 300 amino acid N-terminal FERM domain can bind directly or indirectly to the plasma membrane. ERM proteins are known to be involved in the morphogenesis of specialized membrane structures and in the regulation of cell-cell and cell-matrix adhesion. Activation of ERM proteins, resulting in the unfolding of these proteins, can be performed by single phosphorylation of a conserved C-terminal Thr residue (located at position 558 in moesin), and is induced by PKC-8 in vitro and RhoA- and Rho-kinase in vivo. Ariel et al., J. Immunol. 166:3052-3060 (2001); Chowers, et al., Gastroenterology, 120:449-(2001); Hershkoviz et al., Immunol. 99:87-(2000).

Moesin has been found in epithelial cells, lymphocytes, endothelial cells, and certain types of tumor cells. While traditionally reported to be located in the cytoplasm or the interior face of the plasma membrane, growing evidence now indicates that moesin may also be found on the surface of certain cell types. For example, moesin was found to be expressed on the surface of HT-29 and Caco-2 human epithelial cell lines, as well as the U-937 human monocyte cell line and PBMC. It has also been shown that cell surface-expressed moesin interacts physically and functionally with heparan sulfate, LPS, and components of the measles virus, and was proposed to function as, or be associated with, a cellular receptor for these ligands. The activation of T cells by different physiological and pharmacological agents, such as PHA, PMA, and superantigens, leads to increased expression of surface molecules, such as IL-2Ra, CD69, and other receptors. Toxic shock syndrome toxin (TSST-I) is a staphylococcal enterotoxin that binds the β chain of the TcR and functions as a superantigen. As a consequence, TSST-1 induces the proliferation of T cells in atopic eczema, induces TNFα, interleukin (IL)-1, IL-6 and IL-2, and IFNγ secretion from PBMC and increases the expression of pro-inflammatory receptors, such as chemokine receptors and E-selectin ligand on T cells.

### SUMMARY OF THE INVENTION

The background art does not teach or suggest a method for inhibiting cytokine secretion through binding of a disaccharide to moesin. The background art also does not teach or suggest treating a malignancy or an inflammatory condition by administering a substance that is capable of mediating an activity through moesin.

Moreover, the background art also does not teach or suggest inhibiting viral, bacterial or parasitic infection through binding of a disaccharide to moesin. The background art also does not teach or suggest treating injured nerve growth or regeneration, hippocampal and cortical neuronal regeneration, CNS inflammatory disease, injury or scar formation.

The present invention overcomes these deficiencies of the background art by providing a method for inhibiting inflammatory, cell migration or cell adhesion effects through mediating modulation of the activity of moesin, in which the activity is capable of being mediated, and more preferably activated or reduced, through binding of a saccharide, particularly a disaccharide, to moesin.

It was shown that disaccharide molecules derived from heparin and from heparan sulfate can inhibit the secretion of cytokines such as IL-8 and IL-1β, which activate or induce inflammatory, cell migration or cell adhesion activities. These disaccharide molecules show a dose-dependent inhibition of both spontaneous and TNFα-stimulated cytokine secretion. As described in greater detail below, these effects are mediated through moesin, and are blocked by antagonists such as anti-moesin specific antibodies that bind to meosin.

Although this embodiment of the subject invention centers around moesin, it should be noted that subject invention encompasses any activity mediated through an ERM protein, as previously described.

### BRIEF DESCRIPTION OF THE DRAWINGS

The invention is herein described, by way of example only, with reference to the accompanying drawings, wherein:

FIG. 1 shows the expression of moesin by HT-29 epithelial cells: A. HT-29 cells were grown to confluence. Following culture, the cells were treated with EDTA and the layers were mechanically disrupted. The cells were stained and subjected to FACS analysis. Staining with a specific anti-moesin monoclonal antibody is shown by the solid line. Staining with an isotype control antibody (anti-CD25) is shown by the dashed line. The control with the second antibody only is shown by the filled histogram. B. HT-29 cells were grown to confluence. Following culture, the cells were treated with trypsin and subjected to staining and FACS analysis as in FIG. 1A.

FIG. 2 shows binding of DS-9392 to immobilized recombinant moesin: Plates were coated with recombinant moesin or purified BSA. Following coating, DS-9392 was added to the plates, incubated and washed. Detection was performed using an anti-heparan sulfate mAb followed by an anti-rat IgM Ab. Each experiment was performed in duplicate. The results represent mean and SD. The difference was significant (P).

FIG. 3 shows the effect of anti-moesin antibodies and DS-9267 on TNFα-induced IL-8 secretion from HT-29 cells: HT-29 cells were grown to confluence. Following culture, the cells were pre-incubated for 30 minutes with either anti-moesin or control antibody (anti HSP-60). Then DS-9267 was added for 30 minutes, after which TNFα (200 ng/ml) was added and the cells were incubated for additional 20 hours. Subsequently, the supernatants were collected and the level of IL-8 was determined.

FIG. 4 shows the effect of co-culture of recombinant moesin with DS-9267 on the secretion of IL-8 and IL-1 from TNFα-induced HT-29 cells: HT-29 cells were grown to confluence. Following culture, the cells were supplemented with fresh medium and the DS-9267 which was pre-incubated for 30 minutes with recombinant moesin at the indicated concentrations was added to all cells except for the controls. The culture was continued for 24 hours. Subsequently, the supernatants were collected and assayed for the concentrations of IL-8 (A) and IL-1β (B).

FIG. 5 shows the effect of anti-moesin antibodies on DS-9267-induced Jurkat cell adhesion to fibronectin: Jurkat cells were labeled with ⁵¹[Cr], pretreated (for 30 minutes at 4° C.) with several concentrations of anti-moesin antibody and then added with DS-9267 (100 ng/ml) to microtitre wells that had been pre-coated with fibronectin (1 μg/ml). The amount of adherent cells was determined 30 minutes later. Non-adherent cells were washed away and the remaining bound cells were lysed. The radioactivity of lysates, representing the amount of fibronectin-adherent cells, was determined using a γ-counter. The results represent the percentage of cells that were bound to the wells from total cells that were added to each well.

FIG. 6 shows the effect of recombinant moesin on DS-9267-induced Jurkat cell adhesion to fibronectin: Jurkat cells were labeled with ⁵¹[Cr] and then added with DS-9267 that were pre-incubated (30 min, 4° C.) with several concentrations of recombinant moesin, to microtitre wells that had been pre-coated with fibronectin (1 μg/ml). The amount of adherent cells was determined 30 minutes later. Non-adherent cells were washed away and the remaining bound cells were lysed. The radioactivity of lysates, representing the amount of fibronectin-adherent cells, was determined using a γ-counter. The results represent the percentage of cells that were bound to the wells from total cells that were added to each well.

FIG. 7 shows that DS9392 and DS9267 pretreatment of T cells, specifically inhibits chemokine-mediated T cell adhesion. T cells were pretreated with DS9392 (A), DS9267 or DS8892 (B), at 1 ng/ml, 30 minutes incubation for each, and were then seeded on fibronectin (FN)-coated microtiter wells and activated with either PMA (50 ng/ml), anti-CD3 mAb (15 μg/ml), IL-2 (10 IU/ml) or one of the chemoattractants, MIP-Iβ, SDF-Iα or RANTES (20 ng/ml each). T cell adhesion was then measured.

### DESCRIPTION OF THE PREFERRED EMBODIMENTS

The present invention is of a method for inhibiting inflammatory, cell migration or cell adhesion effects through mediating an activity of moesin in which the activity is capable of being mediated and, more preferably, activated through binding of a saccharide, particularly a disaccharide or a derivative thereof to moesin.

Moesin and other ERM proteins have been implicated in a variety of biological activities and conditions including heart disease such as coronary arteriosclerosis (Morishige et al., Arteriosclerosis, Thromsosis, and Vasc. Bio., 21:548 (2001)); cancers such as breast cancer (Carmeci et al., Surgery, 124:211 (1998)), CNS cancers such as glioma and glial harmartoma (Stemmer-Racjamimov et al., J Neuropathol Exp Neurol, 56:735 (1997)), liver cancer (hepatocellular carcinoma; Guan et al., Ai Zheng, 21:281 (2002)), lung cancer (adenocarcinoma, Tokunou et al., Lab Invest., 80:1643 (2000)), head and neck cancer (epithelial dysplasia, verrucous carcinoma, oral squamous cell carcinoma, Kobayashi et al., J Oral Pathol Med, 32:344 (2003); Kobayashi et al., Clin Cancer Res, 10:572 (2004)), skin cancer (melanocytic, Ichikawa et al., Br J Dermatol, 138:763 (1998); epithelial skin tumors, Ichikawa, J Cutan Pathol, 25:237 (1998)); pancreatic cancer (pancreatic adenocarcinoma, Akisawa et al., Bioch Biophys Res Commun., 258:395 (1999)), prostate cancer (Harrison et al., Int J Oncol, 21:935 (2002)), stomach cancer (Selbach et al., Proteomics, 4:2961 (2004)); metastatic cancer (Martin et al., Crit Rev Oncol Hematol, 46:165 (2003)); nerve growth and regeneration (Olsson et al., J Biol Chem, 254:36288 (1999); hippocampal and cortical neuronal regeneration, Haas et al., Eur J Neurosci., 20:1436 (2004)); CNS inflammatory disease, injury and scar formation (John et al., J Neurosci, 24:2837 (2004)); Down's syndrome (Lubec et al., Bioch Biophys Res Commun., 286:1191 (2001)); bacterial infections such as Helicobacter pylori (Selbach et al., supra), streptococcus (Hoe et al., PNAS, 99:7646 (2002)), shigella (Skoudy et al., J of Cell Sci, 112:2059 (1999)), Neisseria meningitides, Eugene et al, J of Cell Sci, 115:1231 (2002) and Pseudomonas aeruginosa, Maresso et al, J Biol Chem, 279:38402 (2004); viruses such as measles (Blau and Compans, Virology, 210:91 (1995), HIV (Hecker et al., Virus Res, 49:215 (1997) hepatitis virus such as hepatitis B (Lara-Pezzi et al., Hepatology, 33:1270 (2001) and rabies (Sagara et al., Virology, 206:485 (1995)); GI tract conditions such as gastric ulcer and gastiritis (Selbach et al., supra); and skin diseases such as psoriasis (Helms et al., Nat Genet, 35:299 (2003)).

Therefore, as described in detail below, the methods and compounds of the subject invention can be used to prevent or treat the above-described conditions. Additional conditions are described below.

According to an embodiment of the present invention, there is provided a method for inhibiting chemokine-dependent migration or adhesion of cells expressing moesin, comprising mediating the inhibition of the chemokine-dependent activity through at least one activation of moesin or at least one modification of existing moesin activity. Preferably, the cells comprise at least one immune or immune-related cells, or tumor or malignant cells. Also preferably, activation or modification of moesin activity comprises modification potentially mediated through binding of a sulfated saccharide or derivative thereof to moesin. More preferably, the method includes administering a sulfated saccharide or a derivative thereof to a subject. Also more preferably, the method includes administering an antagonist for blocking binding of an activating substance to moesin, wherein said activating substance activates or modulates moesin through a mechanism that can be mediated through binding of a sulfated saccharide to moesin.

According to another embodiment of the present invention, there is provided a method for diminishing induced moesin-mediated intracellular signaling, wherein the signaling is capable of being mediated through an effect of a saccharide binding to moesin, comprising altering moesin activity in cells such that the moesin-mediated intracellular signaling is reduced, wherein the moesin activity is characterized by being capable of being mediated through the effect of the saccharide.

Preferably, the saccharide comprises a heparin/heparan sulfate-derived saccharide or derivative thereof. More preferably, the saccharide or derivative thereof is sulfated. Even more preferably, the saccharide comprises a disaccharide or derivative thereof. Yet more preferably, the saccharide comprises or consists of DS-9267 or DS-9392. Optionally and preferably, the moesin activity is altered through administration of the saccharide or derivative thereof to a subject.

According to still another embodiment of the present invention, there is provided a method for modifying at least one effect of at least one external influence on an eukaryotic cell, wherein the at least one effect is affected by binding of a saccharide to moesin, comprising binding of the saccharide to moesin, thereby modifying the effect. The term “affected” means increased or reduced.

According to yet another method of the present invention, there is provided a method for modifying at least one effect of at least one external influence on an eukaryotic cell, wherein the at least one effect is mediated by binding of a saccharide to moesin, comprising altering the at least one effect by binding a substance to moesin, thereby modifying the effect. Preferably, the substance comprises a saccharide-like molecule or molecules, or a saccharide homolog or analog or derivative. More preferably, the substance comprises a material having a saccharide-like effect.

The subject invention also provides a method of improving, preventing or treating a condition.

More preferably, the condition is measles infection, rabies infection, adenovirus infection, parasitic infection, bacterial infection, nerve injury or damage, central nervous system (CNS) inflammatory disease, brain injury, lung cancer, CNS cancer, head and neck cancer, skin cancer, pancreatic cancer, metastatic cancer, GI cancer, GI disease, skin disease, metastasis in various cancers or nerve regeneration. The method comprises administering a compound of the formula:

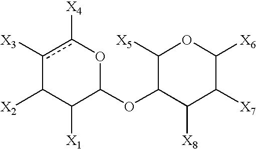

wherein:

the dotted line is an optional double bond;

X₁ is hydroxyl, C₁ to C₁₂ alkoxy, C₁ to C₁₂ substituted alkoxy, sulfate, amino, (monosubstituted) amino or (disubstituted) amino;

X₂ is hydroxyl, C₁ to C₁₂ alkoxy or C₁ to C₁₂ substituted alkoxy;

X₃ is hydrogen, hydroxyl, C₁ to C₁₂ alkoxy or C₁ to C₁₂ substituted alkoxy;

X₄ is C₁ to C₁₂ alkyl, C₁ to C₁₂ substituted alkyl, hydrogen or the formula —C(O)OR, wherein R is absent or is C₁ to C₁₂ alkyl, C₁ to C₁₂ substituted alkyl or hydrogen;

X₅ is C₁ to C₁₂ alkyl, C₁ to C₁₂ substituted alkyl, C₁ to C₁₂ alkoxycarbonyl or C₁ to C₁₂ substituted alkoxycarbonyl;

X₆ is hydroxyl, C₁ to C₁₂ alkoxy or C₁ to C₁₂ substituted alkoxy;

X₇ is hydroxyl, C₁ to C₁₂ alkoxy, C₁ to C₁₂ substituted alkoxy, sulfate, amino, (monosubstituted) amino or (disubstituted) amino; and

X₈ is hydroxyl, C₁ to C₁₂ alkoxy or C₁ to C₁₂ substituted alkoxy.

In a more preferred embodiment, X₁—OH, —OSO₃H, —OSO₃⁻, —NHSO₃H or —NHSO₃⁻; X₂ is —OH; X₃ is —OH or hydrogen; X₄ is —CH₂OSO₃H, —CH₂OSO₃⁻, —C(O)O⁻, —C(O)OH or hydrogen; X₅ is —CH₂OH, —CH₂OSO₃H or CO₂H; X₆ is —OH; X₇ is —OSO₃H, —OSO₃⁻, —NHSO₃H, —NHSO₃⁻, —NHC(O)CH₃, —NH₂ or —NH₃⁺; and X₈ is —OH.

In another preferred embodiment of the invention, the compound has the formula:

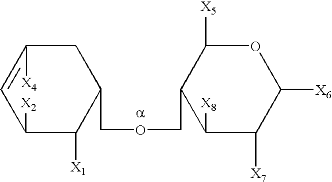

wherein:

X₁ is hydroxyl, C₁ to C₁₂ alkoxy, C₁ to C₁₂ substituted alkoxy, sulfate, amino, (monosubstituted) amino or (disubstituted) amino;

X₂ is hydroxyl, C₁ to C₁₂ alkoxy or C₁ to C₁₂ substituted alkoxy;

X₃ is hydrogen, hydroxyl, C₁ to C₁₂ alkoxy or C₁ to C₁₂ substituted alkoxy;

X₄ is C₁ to C₁₂ alkyl, C₁ to C₁₂ substituted alkyl, hydrogen or the formula —C(O)OR, wherein R is absent or is C₁ to C₁₂ alkyl, C₁ to C₁₂ substituted alkyl or hydrogen;

X₅ is C₁ to C₁₂ alkyl, C₁ to C₁₂ substituted alkyl, C₁ to C₁₂ alkoxycarbonyl or C₁ to C₁₂ substituted alkoxycarbonyl;

X₆ is hydroxyl, C₁ to C₁₂ alkoxy or C₁ to C₁₂ substituted alkoxy;

X₇ is hydroxyl, C₁ to C₁₂ alkoxy, C₁ to C₁₂ substituted alkoxy, sulfate, amino, (monosubstituted) amino or (disubstituted) amino; and

X₈ is hydroxyl, C₁ to C₁₂ alkoxy or C₁ to C₁₂ substituted alkoxy.

In a further preferred embodiment, X₁ is —OH, —OSO₃H, —OSO₃⁻, —NHSO₃H or —NHSO₃⁻; X₂ is —OH; X₄ is —CH₂OSO₃H, —CH₂OSO₃⁻, —C(O)O⁻, —C(O)OH or hydrogen; X₅ is —CH₂OH, —CH₂OSO₃H, —CH₂OSO₃⁻, —C(O)O⁻ or —C(O)OH; X₆ is —OH; X₇ is —OSO₃H, —OSO₃⁻, —NHSO₃H, —NHSO₃⁻, —NHC(O)CH₃, —NH₂ or —NH₃⁺; and X₈ is —OH.

In an even more preferred embodiment, X₁ is —OSO₃⁻; X₂ is —OH; X₄ is —C(O)O⁻; X₅ is —CH₂OSO₃⁻; X₆ is —OH; X₇ is —NHSO₃⁻; and X₈ is —OH. This is DS-9267.

In another preferred embodiment, X₁ is —OSO₃⁻; X₂ is —OH; X₄ is —C(O)O⁻; X₅ is —CH₂OH; X₆ is —OH; X₇ is —NHSO₃⁻; and X₈ is —OH. This is DS-9392.

In yet another preferred embodiment of the invention, the condition is measles infection, rabies infection, adenovirus infection, parasitic infection, bacteria infection, nerve injury or damage, central nervous system (CNS) inflammatory disease, brain injury, lung cancer, CNS cancer, head and neck cancer, skin cancer, pancreatic cancer, metastatic cancer, skin disease, metastasis in various cancers or nerve regeneration. In another embodiment of the invention, the condition is inflammation in general, allergy, cancer in general, other viral infections or autoimmune diseases.

In another aspect of the invention, a method is provided for inhibiting chemokine-dependent migration or chemokine-dependent adhesion of cells expressing moesin, comprising mediating the inhibition of the chemokine-dependent activity through at least one modification of moesin or at least one modification of existing moesin activity. Preferably, the cells include immune, immune-related, tumor or malignant cells.

The modification of moesin activity can include a modification that can be mediated through binding of a saccharide to meosin. Preferably, the saccharide is sulfated. Also preferably, the saccharide is a disaccharide and, more preferably, sulfated.

In the above-described method, a disaccharide or a derivative thereof can be administered to a subject. More preferably, the disaccharide or derivative thereof has the formula:

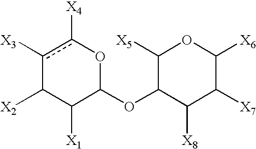

with the variables as described above.

Even more preferably, the disaccharide or derivative thereof has the formula:

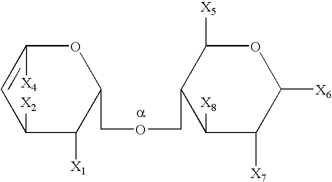

with the variables as described above.

Also provided herein is a method for increasing or reducing moesin-mediated intracellular signaling, wherein said signaling is capable of being mediated through an effect of a saccharide binding to moesin, comprising altering moesin activity in cells such that the moesin-mediated intracellular signaling is increased or reduced. The moesin activity can be altered through administration of a saccharide or derivative thereof.

The saccharide or derivative thereof can be derived from heparin or heparan sulfate. The saccharide or derivative thereof can be sulfated. The saccharide or derivative thereof can be a disaccharide. The disaccharide or derivative thereof can have the formula:

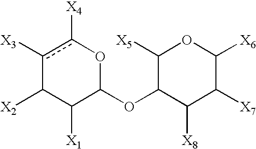

with the variables as described above.

The disaccharide or derivative thereof can also have the formula:

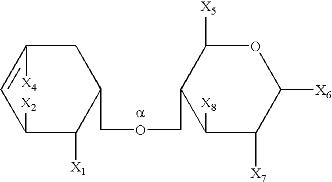

with the variables as described above.

The subject invention further provides a method for modifying at least one effect of at least one external influence on an eukaryotic cell, wherein the at least one effect is affected by binding of a saccharide to moesin, thereby modifying the effect. The effect can be increased or decreased.

The invention also provides a method for modifying at least one effect of at least one external influence on an eukaryotic cell, wherein the at least one effect is mediated by binding of a saccharide to moesin, comprising altering the at least one effect by binding a substance to meosin, thereby modifying the effect. The saccharide or derivative thereof can be derived from heparin or heparan sulfate. The saccharide or derivative thereof can be sulfated, and can be a disaccharide.

More particularly, the disaccharide or derivative thereof can have the formula:

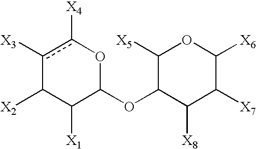

wherein the variables are as described above.

Even more particularly, the disaccharide or derivative thereof can have the formula:

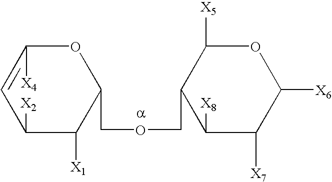

wherein the variables are as described above.

The invention further provides a method for blocking cell migration or adhesion, comprising administering an activity modulating agent capable of mimicking binding of a saccharide to moesin, wherein the cell migration or adhesion is capable of being blocked by a saccharide binding to said moesin. The modulating agent can be administered to treat a disease that is mediated by cell migration or adhesion. The modulating agent can be administered to treat a disease characterized by malignant cell growth.

Also provided herein is a method for blocking cytokine secretion, comprising administering an activating agent for activating moesin through a mechanism activated by saccharide binding to moesin. The activating agent can be used to treat a disease mediated through a cytokine.

According to still another embodiment of the present invention, there is provided a method for blocking cell migration or adhesion, comprising administering a blocking agent capable of mimicking binding of a saccharide to moesin, wherein the cell migration and/or adhesion is capable of being blocked by a saccharide binding to the moesin.

The present invention also encompasses methods for treating a disease mediated by cell migration or adhesion, comprising administering a blocking agent that is capable of mimicking binding of a saccharide to moesin to treat the disease. Other treatable diseases according to the present invention include diseases mediated through a cytokine, comprising administering an activating agent for activating moesin through a mechanism activated by saccharide binding to moesin; and diseases characterized by malignant cell growth, comprising administering a blocking agent that is capable of mimicking binding of a saccharide to moesin.

Moesin is expressed inside the cells and on the cell surface, where it binds to sulfated disaccharides. These sulfated disaccharides bind to moesin and modify its activity, and thereby have a number of effects on the cell. Blocking binding to moesin, blocks these effects. The effects of moesin-binding include inhibition of cytokine secretion (both spontaneous and induced by cytokine such as TNF-α); induction of adhesion of human T cells to ECM (extra cellular matrix); and activation of signaling pathways such as pyk-2 but not ERK pathways. Pre-incubation of cells with sulfated disaccharides inhibits the response of cells to chamomiles, thereby blocking both chemokine mediated adhesion and migration.

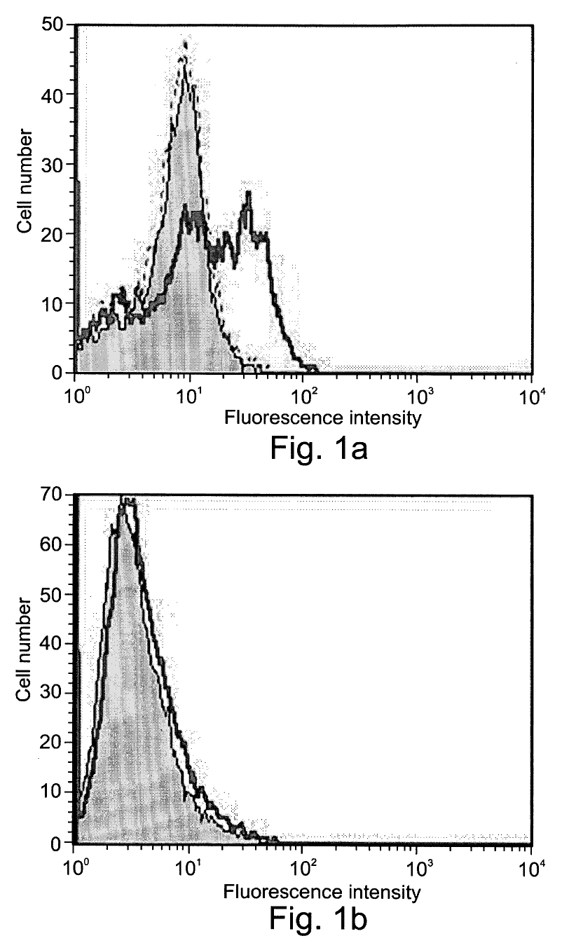

Moesin has been detected on the surface of freshly isolated human peripheral blood T cells. Moesin may have a role in the regulation of T cell adhesion to extra cellular matrix (ECM) components in general, and as a receptor for an adhesion-modulating IL-2-derived peptide (4). Moesin was found to be expressed on human intestinal epithelial (HT-29) cells. As shown in FIG. 1 A, FACS analysis revealed that HT-29 cells were stained positively for moesin. FIG. 1 B shows that the expression of moesin was abolished following mild treatment of the cells with trypsin. These findings indicate that moesin is expressed on the cell-surface of gut epithelial cells and T cells.

The compounds of the present invention can be made by methods known in the art, including those described in U.S. Pat. No. 5,861,382. Examples of such compounds include (DS-9392) 2-O-Sulfate-4-deoxy-4-en-iduronic acid-(alpha-1,4)-2-deoxy-2-N-sulfateglucosamine; (DS-1020) 4-deoxy-4-en-iduronic acid-(alpha-1,4)-2-deoxy-2-N-sulfate-6-O-sulfateglucosamine; (DS-9267) 2-O-sulfate-4-deoxy-4-en-iduronic acid-(alpha.-1,4)-2-deoxy-2-N-sulfate-6-O-sulfateglucosamine; (DS-9517) 2-O-sulfate-4-deoxy-4-en-iduronic acid-(alpha-1,4)-2-deoxy-2-N-acetyl-6-O-sulfateglucosamine; (DS-0895) 4-deoxy-4-en-iduronic acid-(alpha-1,4)-2-deoxy-2-N-acetylglucosamine; (DS-9017) 4-deoxy-4-en-iduronic acid-(alpha-1,4)-2-deoxy-6-O-sulfateglucosamine; (DS-8642) 4-deoxy-4-en-iduronic acid-(alpha-1,4)-2-deoxy-2-N-acetyl-6-O-sulfateglucosamine; (DS-9142) 2-O-sulfate-4-deoxy-4-en-iduronic acid-(alpha-1,4)-2-deoxyglucosamine; (DS-8767) 2-O-sulfate-4-deoxy-4-en-iduronic acid-(alpha-1,4)-2-deoxy-2-N-acetylglucosamine; (DS-8892) 2-O-sulfate-4-deoxy-4-en-iduronic acid-(alpha-1,4)-2-deoxy-6-O-sulfateglucosamine; and (DS-1145) 4-deoxy-4-en-iduronic acid-(alpha-1,4)-2-deoxy-2-N-sulfateglucosamine.

The invention further provides use of the compounds disclosed herein for the treatment of the indications disclosed herein. Moreover, the invention provides use of the compounds disclosed herein for the preparation of medicaments for the treatment of the indications disclosed herein.

When the above-described compounds include one or more choral centers, the stereochemistry of such choral centers can independently be in the R or S configuration, or a mixture of the two. The choral centers can be further designated as R or S or R, S or did, loll or dell, D, L.

Regarding the compounds and combinatorial libraries described herein, the suffix “erne” added to any of the described terms means that two parts of the subsistent are each connected to two other parts in the compound (unless the subsistent contains only one carbon, in which case such carbon is connected to two other parts in the compound, for example, ethylene).

The term “C₁ to C₁₂ alkyl” denotes such radicals as methyl, ethyl, n-propel, isopropyl, n-butyl, is-butyl, sec-butyl, tart-butyl, amyl, tart-amyl, hexyls, hefty, octal, only, decyl, undecyl, dodecyl and the like. Preferred “C₁ to C₁₂ alkyl” groups are methyl, ethyl, iso-butyl, sec-butyl and iso-propyl. Similarly, the term “C₁ to C₁₂ alkylene” denotes radicals of 1 to 12 carbons connected to two other parts in the compound.

The term “C₂ to C₁₂ alkenyl” denotes such radicals as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl, (as well as octenyl, nonenyl, decenyl, undecenyl, dodecenyl radicals attached at any appropriate carbon position and the like) as well as dienes and trienes of straight and branched chains.

The term “C₂ to C₁₂ alkynyl” denotes such radicals as ethanol, propynyl, 2-butynyl, 2-pentynyl, 3-pentynyl, 2-hexynyl, 3-hexynyl, 4-hexynyl, 2-heptynyl, 3-heptynyl, 4-heptynyl, 5-heptynyl (as well as octynyl, nonynyl, decynyl, undecynyl, dodecynyl radicals attached at any appropriate carbon position and the like) as well as di- and tri-ynes of straight and branched chains.

The terms “C₁ to C₁₂ substituted alkyl,” “C₂ to C₁₂ substituted alkenyl,” “C₂ to C₁₂ substituted alkynyl,” “C₁ to C₁₂ substituted alkylene,” “C₂ to C₁₂ substituted alkenylene” and “C₂ to C₁₂ substituted alkynylene” denote groups are substituted by one or more, and preferably one or two, halogen, hydroxy, protected hydroxy, oxo, protected oxo, C₃ to C₇ cycloalkyl, phenyl, naphthyl, amino, protected amino, (monosubstituted)amino, protected (monosubstituted)amino, (disubstituted)amino, guanidino, protected guanidino, heterocyclic ring, substituted heterocyclic ring, imidazolyl, indolyl, pyrrolidinyl, C₁ to C₁₂ alkoxy, C₁ to C₁₂ acyl, C₁ to C₁₂ acyloxy, nitro, carboxy, protected carboxy, carbamoyl, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, cyano, methylsulfonylamino, sulfate, thiol, C₁ to C₁₀ alkylthio or C₁ to C₁₀ alkylsulfonyl groups. The substituted alkyl groups may be substituted once or more, and preferably once or twice, with the same or with different substituents.

The term “protected oxo” denotes a carbon atom bonded to two additional carbon atoms substituted with two alkoxy groups or twice bonded to a substituted diol moiety, thereby forming an acyclic or cyclic ketal moiety.

The term “C₁ to C₁₂ alkoxy” as used herein denotes groups such as methoxy, ethoxy, n-propoxy, isopropoxy, n-butoxy, t-butoxy and like groups. A preferred alkoxy is methoxy. The term “C₁ to C₁₂ substituted alkoxy” means the alkyl portion of the alkoxy can be substituted in the same manner as in relation to C₁ to C₁₂ substituted alkyl. Similarly, the term “C₁ to C₁₂ phenylalkoxy” as used herein means “C₁ to C₁₂ alkoxy” bonded to a phenyl radical.

The term “C₁ to C₁₂ acyloxy” denotes herein groups such as formyloxy, acetoxy, propionyloxy, butyryloxy, pivaloyloxy, pentanoyloxy, hexanoyloxy, heptanoyloxy, octanoyloxy, nonanoyloxy, decanoyloxy, undecanoyloxy, dodecanoyloxy and the like.

Similarly, the term “C₁ to C₁₂ acyl” encompasses groups such as formyl, acetyl, propionyl, butyryl, pentanoyl, pivaloyl, hexanoyl, heptanoyl, octanoyl, nonanoyl, decanoyl, undecanoyl, dodecanoyl, benzoyl and the like. Preferred acyl groups are acetyl and benzoyl.

The term “C₁ to C₁₂ substituted acyl” denotes the acyl group substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₁₂ alkoxy, C₁ to C₁₂ acyl, C₁ to C₁₂ acyloxy, nitro, C₁ to C₁₂ alkyl ester, carboxy, protected carboxy, carbamoyl, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, cyano, methylsulfonylamino, thiol, C₁ to C₁₀ alkylthio or C₁ to C₁₀ alkylsulfonyl groups. The substituted acyl groups may be substituted once or more, and preferably once or twice, with the same or with different substituents.

The term “C₃ to C₇ substituted cycloalkyl” or “C₅ to C₇ substituted cycloalkyl” indicates the above cycloalkyl rings substituted by one or two halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino groups.

The term “cycloalkylene” means a cycloalkyl, as defined above, where the cycloalkyl radical is bonded at two positions connecting together two separate additional groups. Similarly, the term “substituted cycloalkylene” means a cycloalkylene where the cycloalkyl radical is bonded at two positions connecting together two separate additional groups and further bearing at least one additional substituent.

The term “substituted C₅ to C₇ cycloalkenylene” means a cycloalkenylene further substituted by halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino group.

The term “heterocycle” or “heterocyclic ring” denotes optionally substituted five-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings may be saturated, fully unsaturated or partially unsaturated, with fully saturated rings being preferred. Preferred heterocyclic rings include morpholino, piperidinyl, piperazinyl, 2-amino-imidazoyl, tetrahydrofurano, pyrrolo, tetrahydrothiophen-yl, hexylmethyleneimino and heptylmethyleneimino.

The term “substituted heterocycle” or “substituted heterocyclic ring” means the above-described heterocyclic ring is substituted with, for example, one or more, and preferably one or two, substituents which are the same or different which substituents can be halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino, N-(phenylsulfonyl)amino, heterocycle or substituted heterocycle groups.

The term “heteroaryl” means a heterocyclic aromatic derivative which is a five-membered or six-membered ring system having from 1 to 4 heteroatoms, such as oxygen, sulfur and/or nitrogen, in particular nitrogen, either alone or in conjunction with sulfur or oxygen ring atoms. Examples of heteroaryls include pyridinyl, pyrimidinyl, and pyrazinyl, pyridazinyl, pyrrolo, furano, oxazolo, isoxazolo, phthalimido, thiazolo and the like.

The term “substituted heteroaryl” means the above-described heteroaryl is substituted with, for example, one or more, and preferably one or two, substituents which are the same or different which substituents can be halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino or N-(phenylsulfonyl)amino groups.

The term “C₇ to C₁₈ phenylalkyl” denotes a C₁ to C₁₂ alkyl group substituted at any position within the alkyl chain by a phenyl. The definition includes groups of the formula: -phenyl-alkyl, -alkyl-phenyl and -alkyl-phenyl-alkyl.

Similarly, the term “C₁ to C₁₂ heterocycloalkyl” denotes a C₁ to C₁₂ alkyl group substituted at any position within the alkyl chain by a “heterocycle,” as defined herein. The definition includes groups of the formula: -heterocyclic-alkyl, -alkyl-heterocyclic and -alkyl-heterocyclic-alkyl. Examples of such a group include 2-pyridylethyl, 3-piperydyl(n-propyl), 4-furylhexyl, 3-piperazyl(n-amyl), 3-morpholyl(sec-butyl) and the like. Preferred C₁ to C₁₂ heterocycloalkyl groups are any one of the preferred alkyl groups described herein combined with any one of the preferred heterocycle groups described herein.

The terms “C₇ to C₁₈ substituted phenylalkyl” and “C₁ to C₁₂ substituted heterocycloalkyl” denote a C₇ to C₁₈ phenylalkyl group or C₁ to C₁₂ heterocycloalkyl substituted (on the alkyl or, where applicable, phenyl or heterocyclic portion) with one or more, and preferably one or two, groups chosen from halogen, hydroxy, protected hydroxy, oxo, protected oxo, amino, protected amino, (monosubstituted)amino, protected (monosubstituted)amino, (disubstituted)amino, guanidino, protected guanidino, heterocyclic ring, substituted heterocyclic ring, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, nitro, carboxy, protected carboxy, carbamoyl, carboxamide, protected carboxamide, N—(C₁ to C₁₂ alkyl)carboxamide, protected N—(C₁ to C₁₂ alkyl)carboxamide, N,N—(C₁ to C₁₂ dialkyl)carboxamide, cyano, N—(C₁ to C₁₂ alkylsulfonyl)amino, thiol, C₁ to C₁₀ alkylthio, C₁ to C₁₀ alkylsulfonyl groups; and/or the phenyl group may be substituted with one or more, and preferably one or two, substituents chosen from halogen, hydroxy, protected hydroxy, cyano, nitro, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino, N-(phenylsulfonyl)amino, cyclic C₂ to C₁₂ alkylene or a phenyl group, substituted or unsubstituted, for a resulting biphenyl group. The substituted alkyl, phenyl or heterocyclic groups may be substituted with one or more, and preferably one or two, substituents which can be the same or different.

The term “C₇ to C₁₈ phenylalkylene” specifies a C₇ to C₁₈ phenylalkyl, as defined above, where the phenylalkyl radical is bonded at two different positions connecting together two separate additional groups. The definition includes groups of the formula: -phenyl-alkyl-, -alkyl-phenyl- and -alkyl-phenyl-alkyl-. Substitutions on the phenyl ring can be 1,2, 1,3 or 1,4.

C₇ to C₁₈ phenylalkylenes include, for example, 1,4-tolylene and 1,3-xylylene.

Similarly, the term “C₁ to C₁₂ heterocycloalkylene” specifies a C₁ to C₁₂ heterocycloalkyl, as defined above, where the heterocycloalkyl radical is bonded at two different positions connecting together two separate additional groups. The definition includes groups of the formula: -heterocyclic-alkyl-, -alkyl-heterocyclic and -alkyl-heterocyclic-alkyl-.

The terms “C₇ to C₁₈ substituted phenylalkylene” and “C₁ to C₁₂ substituted heterocycloalkylene” means a C₇ to C₁₈ phenylalkylene or C₁ to C₁₂ heterocycloalkylene as defined above that is further substituted by halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino group on the phenyl ring or on the alkyl group.

The term “substituted phenyl” specifies a phenyl group substituted with one or more, and preferably one or two, moieties chosen from the groups consisting of halogen, hydroxy, protected hydroxy, cyano, nitro, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino, N-(phenylsulfonyl)amino or phenyl, wherein the phenyl is substituted or unsubstituted, such that, for example, a biphenyl results.

The term “phenoxy” denotes a phenyl bonded to an oxygen atom, wherein the binding to the rest of the molecule is through the oxygen atom. The term “substituted phenoxy” specifies a phenoxy group substituted with one or more, and preferably one or two, moieties chosen from the groups consisting of halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino and N-(phenylsulfonyl)amino.

The term “C₇ to C₁₈ substituted phenylalkoxy” denotes a C₇ to C₁₈ phenylalkoxy group bonded to the rest of the molecule through the oxygen atom, wherein the phenylalkyl portion is substituted with one or more, and preferably one or two, groups selected from halogen, hydroxy, protected hydroxy, oxo, protected oxo, amino, protected amino, (monosubstituted)amino, protected (monosubstituted)amino, (disubstituted)amino, guanidino, heterocyclic ring, substituted heterocyclic ring, C₁ to C₁₂ alkoxy, C₁ to C₁₂ acyl, C₁ to C₁₂ acyloxy, nitro, carboxy, protected carboxy, carbamoyl, carboxamide, protected carboxamide, N—(C₁ to C₁₂ alkyl)carboxamide, protected N—(C₁ to C₁₂ alkyl)carboxamide, N,N—(C₁ to C₁₂ dialkyl)carboxamide, cyano, N—(C₁ to C₁₂ alkylsulfonyl)amino, thiol, C₁ to C₁₀ alkylthio, C₁ to C₁₀ alkylsulfonyl groups; and/or the phenyl group can be substituted with one or more, and preferably one or two, substituents chosen from halogen, hydroxy, protected hydroxy, cyano, nitro, C₁ to C₁₂ alkyl, C₁ to C₁₂ alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl) carboxamide, protected N—(C₁ to C₁₂ alkyl) carboxamide, N,N-di(C₁ to C₁₂ alkyl)carboxamide, trifluoromethyl, N—((C₁ to C₁₂ alkyl)sulfonyl)amino, N-(phenylsulfonyl)amino or a phenyl group, substituted or unsubstituted, for a resulting biphenyl group. The substituted alkyl or phenyl groups may be substituted with one or more, and preferably one or two, substituents which can be the same or different.

The term “substituted naphthyl” specifies a naphthyl group substituted with one or more, and preferably one or two, moieties either on the same ring or on different rings chosen from the groups consisting of halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino or N-(phenylsulfonyl)amino.

The term “naphthylene” means a naphthyl radical bonded at two positions connecting together two separate additional groups. Similarly, the term “substituted napthylene” means a naphthylene group that is further substituted by halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino group.

The terms “halo” and “halogen” refer to the fluoro, chloro, bromo or iodo atoms. There can be one or more halogens, which are the same or different. Preferred halogens are chloro and fluoro.

The term “(monosubstituted)amino” refers to an amino group with one substituent chosen from the group consisting of phenyl, substituted phenyl, C₁ to C₁₂ alkyl, C₁ to C₁₂ substituted alkyl, C₁ to C₁₂ acyl, C₁ to C₁₂ substituted acyl, C₂ to C₁₂ alkenyl, C₂ to C₁₂ substituted alkenyl, C₂ to C₁₂ alkynyl, C₂ to C₁₂ substituted alkynyl, C₇ to C₁₈ phenylalkyl, C₇ to C₁₈ substituted phenylalkyl, sulfate, heterocyclic ring, substituted heterocyclic ring, C₁ to C₁₂ heterocycloalkyl and C₁ to C₁₂ substituted heterocycloalkyl. The (monosubstituted)amino can additionally have an amino-protecting group as encompassed by the term “protected (monosubstituted)amino.”

The term “(disubstituted)amino” refers to an amino group with two substituents chosen from the group consisting of phenyl, substituted phenyl, C₁ to C₁₂ alkyl, C₁ to C₁₂ substituted alkyl, C₁ to C₁₂ acyl, C₂ to C₁₂ alkenyl, C₂ to C₁₂ alkynyl, C₇ to C₁₈ phenylalkyl, C₇ to C₁₈ substituted phenylalkyl, sulfate, C₁ to C₁₂ heterocycloalkyl and C₁ to C₁₂ substituted heterocycloalkyl. The two substituents can be the same or different.

The term “sulfate” means —OSO₃H or —OSO₃⁻. The term “amino” means —NH₂ or —NH₃⁺.

The term “amino-protecting group” as used herein refers to substituents of the amino group commonly employed to block or protect the amino functionality while reacting other functional groups of the molecule. The term “protected (monosubstituted)amino” means there is an amino-protecting group on the monosubstituted amino nitrogen atom. In addition, the term “protected carboxamide” means there is an amino-protecting group on the carboxamide nitrogen. Similarly, the term “protected N—(C₁ to C₁₂ alkyl)carboxamide” means there is an amino-protecting group on the carboxamide nitrogen.

Examples of such amino-protecting groups include the formyl (“For”) group, the trityl group, the phthalimido group, the trichloroacetyl group, the chloroacetyl, bromoacetyl, and iodoacetyl groups, urethane-type blocking groups, such as t-butoxycarbonyl (“Boc”), 2-(4-biphenylyl)propyl-2-oxycarbonyl (“Bpoc”), 2-phenylpropyl-2-oxycarbonyl (“Poc”), 2-(4-xenyl)isopropoxycarbonyl, 1,1-diphenylethyl-1-oxycarbonyl, 1,1-diphenylpropyl-1-oxycarbonyl, 2-(3,5-dimethoxyphenyl)propyl-2-oxycarbonyl (“Ddz”), 2-(p-toluoyl)propyl-2-oxycarbonyl, cyclopentanyloxycarbonyl, 1-methylcyclopentanyloxycarbonyl, cyclohexanyloxy-carbonyl, 1-methylcyclohexanyloxycarbonyl, 2-methylcyclohexanyloxycarbonyl, 2-(4-toluylsulfonyl)-ethoxycarbonyl, 2-(methylsulfonyl)ethoxycarbonyl, 2-(triphenylphosphino)-ethoxycarbonyl, 9-fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2,2-trichloroethoxycarbonyl, 2-ethynyl-2-propoxycarbonyl, cyclopropylmethoxycarbonyl, isobornyloxycarbonyl, 1-piperidyloxycarbonyl, benzyloxycarbonyl (“Cbz”), 4-phenylbenzyloxycarbonyl, 2-methylbenzyloxy-carbonyl, -2,4,5-tetramethylbenzyloxycarbonyl (“Tmz”), 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyl-oxycarbonyl, 4-bromobenzyloxycarbonyl, 3-bromobenzyloxycarbonyl, 4-nitrobenzyloxy-carbonyl, 4-cyanobenzyloxycarbonyl, 4-(decyloxy)benzyloxycarbonyl and the like; the benzoylmethylsulfonyl group, dithiasuccinoyl (“Dts”), the 2-(nitro)phenylsulfenyl group (“Nps”), the diphenyl-phosphine oxide group and like amino-protecting groups. The species of amino-protecting group employed is not critical so long as the derivatized amino group is stable to the conditions of the subsequent reaction(s) and can be removed at the appropriate point without disrupting the remainder of the compounds. Preferred amino-protecting groups are Boc, Cbz and Fmoc. Further examples of amino-protecting groups embraced by the above term are well known in organic synthesis and the peptide art and are described by, for example, T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, each of which is incorporated herein by reference. The related term “protected amino” defines an amino group substituted with an amino-protecting group discussed above.

The term “protected guanidino” as used herein refers to an “amino-protecting group” on one or two of the guanidino nitrogen atoms. Examples of “protected guanidino” groups are described by T. W. Greene and P. G. M. Wuts; M. Bodanzsky; and Stewart and Young, supra.

The term “epimino” means —NH—. The term “substituted epimino” means —N(R)—, where R is a substitution group listed above under the definition of “(monosubstituted)amino.”

The term “C₁ to C₅ alkylene epimino” refers to a one to five carbon alkylene chain with an epimino at any point along the chain. The term “C₁ to C₅ substituted alkylene epimino” refers to a C₁ to C₅ alkylene epimino group that is substituted a) at the epimino position (in the same way as “substituted epimino,” described above); and/or b) at one or more of the alkylene positions (in the same way as “substituted alkylene,” as described above).

The term “thio” refers to —SH or, if between two other groups, —S—. The term “C₁ to C₁₀ alkylene thio” refers to a one to ten carbon alkylene chain with a thio at any point along the chain. The term “C₁ to C₁₀ substituted alkylene thio” refers to a C₁ to C₁₀ alkylene thio group that is substituted at one or more of the alkylene positions (in the same way as “substituted alkylene,” as described above).

The term “sulfonyl” refers to —S(O)₂—. The term “C₁ to C₁₀ alkylene sulfonyl” refers to a one to ten carbon alkylene chain with a sulfonyl at any point along the chain. The term “C₁ to C₁₀ substituted alkylene sulfonyl” refers to a C₁ to C₁₀ alkylene sulfonyl group that is substituted at one or more of the alkylene positions (in the same way as “substituted alkylene,” as described above).

The term “sulfinyl” refers to —S(O)—. The term “C₁ to C₁₀ alkylene sulfinyl” refers to a one to ten carbon alkylene chain with a sulfinyl at any point along the chain. The term “C₁ to C₁₀ substituted alkylene sulfinyl” refers to a C₁ to C₁₀ alkylene sulfinyl group that is substituted at one or more of the alkylene positions (in the same way as “substituted alkylene,” as described above).

The term “oxy” refers to —O—. The terms “C₁ to C₁₀ alkylene oxy,” “C₁ to C₁₀ alkylene dioxy” and “C₁ to C₁₀ alkylene trioxy” refer to a one to ten carbon alkylene chain with, respectively, one, two or three —O— at any point along the chain, provided that no two oxygen atoms are consecutive, and provided that any two oxygen atoms are separated by at least two carbons. The terms “C₁ to C₁₀ substituted alkylene oxy,” “C₁ to C₁₀ substituted alkylene dioxy” and “C₁ to C₁₀ substituted alkylene trioxy” refer, respectfully to “C₁ to C₁₀ alkylene oxy,” “C₁ to C₁₀ alkylene dioxy” and “C₁ to C₁₀ alkylene trioxy” that are substituted at one or more of the alkylene positions (in the same way as “substituted alkylene,” as described above).

The term “thiocarbonyl” refers to —C(S)H or, if between two other groups, —C(S)—. The term “thioester” refers to —C(O)SH or, if between two other groups, —C(O)S—.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxytrityl, 4,4′,4″-trimethoxytrityl, 2-phenylpropyl, trimethylsilyl, t-butyldimethylsilyl, phenacyl, 2,2,2-trichloroethyl, (trimethylsilyl)ethyl, (di(n-butyl)methylsilyl)ethyl, p-toluenesulfonylethyl, 4-nitrobenzylsulfonylethyl, allyl, cinnamyl, 1-(trimethylsilylmethyl)propenyl and like moieties. The species of carboxy-protecting group employed is not critical so long as the derivatized carboxylic acid is stable to the conditions of subsequent reaction(s) and can be removed at the appropriate point without disrupting the remainder of the molecule. Further examples of these groups are found in E. Haslam, “Protective Groups in Organic Chemistry,” J. G. W. McOmie, Ed., Plenum Press, New York, N.Y., 1973, Chapter 5, and T. W. Greene and P. G. M. Wuts, “Protective Groups in Organic Synthesis,” 2nd ed., John Wiley and Sons, New York, N.Y., 1991, Chapter 5, each of which is incorporated herein by reference. A related term is “protected carboxy,” which refers to a carboxy group substituted with one of the above carboxy-protecting groups.

The term “hydroxy-protecting group” refers to readily cleavable groups bonded to hydroxyl groups, such as the tetrahydropyranyl, 2-methoxypropyl, 1-ethoxyethyl, methoxymethyl, 2-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl, 2,2,2-trichloroethoxycarbonyl groups and the like. The species of hydroxy-protecting groups is not critical so long as the derivatized hydroxyl group is stable to the conditions of subsequent reaction(s) and can be removed at the appropriate point without disrupting the remainder of the molecule. Further examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3. Related terms are “protected hydroxy,” and “protected hydroxymethyl” which refer to a hydroxy or hydroxymethyl substituted with one of the above hydroxy-protecting groups.

The term “C₁ to C₁₀ alkylthio” refers to sulfide groups such as methylthio, ethylthio, n-propylthio, isopropylthio, n-butylthio, t-butylthio and like groups.

The term “C₁ to C₁₀ alkylsulfoxide” indicates sulfoxide groups such as methylsulfoxide, ethylsulfoxide, n-propylsulfoxide, isopropylsulfoxide, n-butylsulfoxide, sec-butylsulfoxide and the like. The term “C₁ to C₁₀ alkylsulfonyl” encompasses groups such as methylsulfonyl, ethylsulfonyl, n-propylsulfonyl, isopropylsulfonyl, n-butylsulfonyl, t-butylsulfonyl and the like. it should also be understood that the above thio, sulfoxide or sulfonyl groups can be at any point on the alkyl chain (e.g., 2-methylmercaptoethyl).

The terms “C₁ to C₁₀ substituted alkylthio,” “C₁ to C₁₀ substituted alkylsulfoxide,” and “C₁ to C₁₀ substituted alkylsulfonyl,” denote the C₁ to C₁₀ alkyl portion of these groups may be substituted as described above in relation to “substituted alkyl.”

The terms “phenylthio,” “phenylsulfoxide,” and “phenylsulfonyl” specify a thiol, a sulfoxide, or sulfone, respectively, containing a phenyl group. The terms “substituted phenylthio,” “substituted phenylsulfoxide,” and “substituted phenylsulfonyl” means that the phenyl of these groups can be substituted as described above in relation to “substituted phenyl.”

The term “C₁ to C₁₂ alkylaminocarbonyl” means a C₁ to C₁₂ alkyl attached to a nitrogen of the aminocarbonyl group. Examples of C₁ to C₁₂ alkylaminocarbonyl include methylaminocarbonyl, ethylaminocarbonyl, propylaminocarbonyl and butylaminocarbonyl. The term “C₁ to C₁₂ substituted alkylaminocarbonyl” denotes a substituted alkyl bonded to a nitrogen of the aminocarbonyl group, which alkyl may be substituted as described above in relation to C₁ to C₁₂ substituted alkyl. Examples of C₁ to C₁₂ substituted alkylaminocarbonyl include, for example, methoxymethylaminocarbonyl, 2-chloroethylaminocarbonyl, 2-oxopropylaminocarbonyl and 4-phenylbutylaminocarbonyl.

The term “C₁ to C₁₂ alkoxycarbonyl” means a “C₁ to C₁₂ alkoxy” group attached to a carbonyl group. The term “C₁ to C₁₂ substituted alkoxycarbonyl” denotes a substituted alkoxy bonded to the carbonyl group, which alkoxy may be substituted as described above in relation to “C₁ to C₁₂ substituted alkyl.”

The term “phenylaminocarbonyl” means a phenyl attached to a nitrogen of the aminocarbonyl group. The term “substituted phenylaminocarbonyl” denotes a substituted phenyl bonded to a nitrogen of the aminocarbonyl group, which phenyl may be substituted as described above in relation to substituted phenyl. Examples of substituted phenylaminocarbonyl include 2-chlorophenylaminocarbonyl, 3-chlorophenylaminocarbonyl, 2-nitorphenylaminocarbonyl, 4-biphenylaminocarbonyl, and 4-methoxyphenylaminocarbonyl.

The term “C₁ to C₁₂ alkylaminothiocarbonyl” means a C₁ to C₁₂ alkyl attached to an aminothiocarbonyl group, wherein the alkyl has the same meaning as defined above. Examples of C₁ to C₁₂ alkylaminothiocarbonyl include methylaminothiocarbonyl, ethylaminothiocarbonyl, propylaminothiocarbonyl and butylaminothiocarbonyl.

The term “C₁ to C₁₂ substituted alkylaminothiocarbonyl” denotes a substituted alkyl bonded to an aminothiocarbonyl group, wherein the alkyl may be substituted as described above in relation to C₁ to C₁₂ substituted alkyl.

The term “phenylaminothiocarbonyl” means a phenyl attached to an aminothiocarbonyl group, wherein the phenyl has the same meaning as defined above. The term “substituted phenylaminothiocarbonyl” denotes a substituted phenyl bonded to an aminothiocarbonyl group, wherein phenyl may be substituted as described above in relation to substituted phenyl.

The term “phenylene” means a phenyl group where the phenyl radical is bonded at two positions connecting together two separate additional groups. The term “substituted phenylene” means a phenyl group where the phenyl radical is bonded at two positions connecting together two separate additional groups, wherein the phenyl is substituted as described above in relation to “substituted phenyl.”

The term “substituted C₁ to C₁₂ alkylene” means a C₁ to C₁₂ alkyl group where the alkyl radical is bonded at two positions connecting together two separate additional groups and further bearing an additional substituent. Examples of “substituted C₁ to C₁₂ alkylene” includes aminomethylene, 1-(amino)-1,2-ethyl, 2-(amino)-1,2-ethyl, 1-(acetamido)-1,2-ethyl, 2-(acetamido)-1,2-ethyl, 2-hydroxy-1,1-ethyl, 1-(amino)-1,3-propyl.

The terms “cyclic C₂ to C₇ alkylene,” “substituted cyclic C₂ to C₇ alkylene,” “cyclic C₂ to C₇ heteroalkylene,” and “substituted cyclic C₂ to C₇ heteroalkylene,” defines such a cyclic group bonded (“fused”) to the phenyl radical resulting in a bicyclic ring system. The cyclic group may be saturated or contain one or two double bonds. Furthermore, the cyclic group may have one or two methylene or methine groups replaced by one or two oxygen, nitrogen or sulfur atoms which are the cyclic C₂ to C₇ heteroalkylene.

The cyclic alkylene or heteroalkylene group may be substituted once or twice by the same or different substituents which, if appropriate, can be connected to another part of the compound (e.g., alkylene) selected from the group consisting of the following moieties: hydroxy, protected hydroxy, carboxy, protected carboxy, oxo, protected oxo, C₁ to C₄ acyloxy, formyl, C₁ to C₁₂ acyl, C₁ to C₁₂ alkyl, C₁ to C₇ alkoxy, C₁ to C₁₀ alkylthio, C₁ to C₁₀ alkylsulfoxide, C₁ to C₁₀ alkylsulfonyl, halo, amino, protected amino, (monosubstituted)amino, protected (monosubstituted)amino, (disubstituted)amino, hydroxymethyl or a protected hydroxymethyl.

The cyclic alkylene or heteroalkylene group fused onto the benzene radical can contain two to ten ring members, but it preferably contains three to six members. Examples of such saturated cyclic groups are when the resultant bicyclic ring system is 2,3-dihydro-indanyl and a tetralin ring. When the cyclic groups are unsaturated, examples occur when the resultant bicyclic ring system is a naphthyl ring or indolyl. Examples of fused cyclic groups which each contain one nitrogen atom and one or more double bond, preferably one or two double bonds, are when the benzene radical is fused to a pyridino, pyrano, pyrrolo, pyridinyl, dihydropyrrolo, or dihydropyridinyl ring. Examples of fused cyclic groups which each contain one oxygen atom and one or two double bonds are when the benzene radical ring is fused to a furo, pyrano, dihydrofurano, or dihydropyrano ring. Examples of fused cyclic groups which each have one sulfur atom and contain one or two double bonds are when the benzene radical is fused to a thieno, thiopyrano, dihydrothieno or dihydrothiopyrano ring. Examples of cyclic groups which contain two heteroatoms selected from sulfur and nitrogen and one or two double bonds are when the benzene radical ring is fused to a thiazolo, isothiazolo, dihydrothiazolo or dihydroisothiazolo ring. Examples of cyclic groups which contain two heteroatoms selected from oxygen and nitrogen and one or two double bonds are when the benzene ring is fused to an oxazolo, isoxazolo, dihydrooxazolo or dihydroisoxazolo ring. Examples of cyclic groups which contain two nitrogen heteroatoms and one or two double bonds occur when the benzene ring is fused to a pyrazolo, imidazolo, dihydropyrazolo or dihydroimidazolo ring or pyrazinyl.

The term “carbamoyl” means an —NC(O)— group where the radical is bonded at two positions connecting two separate additional groups.

One or more of the compounds of the invention may be present as a salt. The term “salt” encompasses those salts that form with the carboxylate anions and amine nitrogens and include salts formed with the organic and inorganic anions and cations discussed below. Furthermore, the term includes salts that form by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the alkali and alkaline earth metals, (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge et al., J. Pharm. Sci., 66:1-19 (1977), which is incorporated herein by reference. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group is referred to by this term. For example, a cation for a carboxylate anion will exist when a position is substituted with a (quaternary ammonium)methyl group. A preferred cation for the carboxylate anion is the sodium cation.

The compounds of the invention can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

One or more compounds of the invention can be in the biologically active ester form, such as the non-toxic, metabolically-labile ester-form. Such ester forms induce increased blood levels and prolong the efficacy of the corresponding non-esterified forms of the compounds. Ester groups which can be used include the lower alkoxymethyl groups, for example, methoxymethyl, ethoxymethyl, isopropoxymethyl and the like; the —(C₁ to C₁₂) alkoxyethyl groups, for example methoxyethyl, ethoxyethyl, propoxyethyl, isopropoxyethyl and the like; the 2-oxo-1,3-dioxolen-4-ylmethyl groups, such as 5-methyl-2-oxo-1,3-dioxolen-4-ylmethyl, 5-phenyl-2-oxo-1,3-dioxolen-4-ylmethyl and the like; the C₁ to C₁₀ alkylthiomethyl groups, for example methylthiomethyl, ethylthiomethyl, iso-propylthiomethyl and the like; the acyloxymethyl groups, for example pivaloyloxymethyl, pivaloyloxyethyl, -acetoxymethyl and the like; the ethoxycarbonyl-1-methyl group; the -acetoxyethyl; the 1-(C₁ to C₁₂ alkyloxycarbonyloxy)ethyl groups such as the 1-(ethoxycarbonyloxy)ethyl group; and the 1-(C₁ to C₁₂ alkylaminocarbonyloxy)ethyl groups such as the 1-(methylaminocarbonyloxy)ethyl group.

The term “amino acid” includes any one of the twenty naturally-occurring amino acids or the D-form of any one of the naturally-occurring amino acids. In addition, the term “amino acid” also includes other non-naturally occurring amino acids besides the D-amino acids, which are functional equivalents of the naturally-occurring amino acids. Such non-naturally-occurring amino acids include, for example, norleucine (“Nle”), norvaline (“Nva”), L- or D-naphthalanine, ornithine (“Orn”), homoarginine (homoArg) and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference. Amino acids and amino acid analogs can be purchased commercially (Sigma Chemical Co.; Advanced Chemtech) or synthesized using methods known in the art.

It should be understood that any position of the claimed invention has up to three serial “substitutions.” For example, a “substituted alkyl” that is substituted with a “substituted phenyl” that is, in turn, substituted with a “substituted alkyl” can, in turn, be substituted by one more group and no longer further substituted. However, it should also be understood that the invention contemplates, if appropriate, more than three parallel substitutions. For example, if appropriate, more than three hydrogens on an alkyl moiety may be substituted with any one or more of a variety of groups, including halo and hydroxy.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Effect of Disaccharides on Moesin

Materials and Methods

Cell Lines and Culture

The HT-29 (ATCC HTB38) epithelial cell lines were obtained from the American Type Culture Collection (Rockville. MD). Cells were maintained in culture using DMEM media (Bet Haemek, Israel) supplemented with 10% cosmic calf serum (HyClone Laboratories), 1% glutamine, and 1% penicillin/streptomycin (Bet Haemek, Israel), at 37° C., in an atmosphere of 5% CO₂.

Jurkat cells, a CD4+ T-lymphoma cell line, were maintained in medium consisted of RPMI 1640 (Bet Haemek, Israel), supplemented with 10% cosmic calf serum (HyClone Laboratories), 2 mM L-glutamin and 1% Pen-Strep (Bet Haemek, Israel), at 37° C., in an atmosphere of 5% CO₂.

Human T cells were purified from the peripheral blood of healthy donors. Briefly, human peripheral blood was isolated on Ficoll gradients, washed, resuspended in PBS containing 3% heat-inactivated FCS (Bet Haemek, Israel), and incubated (45 min, 37° C., 7% CO₂-humidified atmosphere) on nylon-wool columns (NovaMed; Jerusalem, Israel). Non-adherent cells were eluted and washed, and platelets were removed by centrifugation (700 rpm, 15 min, 18° C.). Residual monocytes were removed by incubation of the cells on tissue culture plates (2 h, 37° C.), after which non-adherent cells were collected. The CD3⁺ content of these PBLs was >95%.

Disaccharides

Heparin-disaccharides were obtained from Sigma. (DS-9267, DS-9392 and DS-8892).

TNFα and Antibodies

TNFα was obtained from Boehringer Mannheim (Indianapolis, Ind.). Mouse anti-human moesin mAb clone 38/87 was obtained from NeoMarkers (Fremont, Calif.). The LKI (anti-HSP 60) mouse mAbs IgG, supplied by W. Van Eden (Utrecht University, The Netherlands). The anti-heparan-sulfate (HK-249) rat IgM which recognizes the sugar moiety of heparan sulfate proteoglycans, was supplied by Yoshiya Tanaka (University of Occupational and Enviromental Health, Japan).

Bacterial Expression and Purification of Recombinant Human Moesin

The plasmid pGEX-KG-human moesin residues 1-577 (pGhuMo) (provided by Prof. Furthhmayer [Stanford, Calif.]) contains human moesin as a fusion protein to glutathione S-transferase (GST). Escherichia coli (E. coli) bacteria were transformed with pGhuMo and grown in L-broth containing penicillin (100 μg/ml). These bacteria were induced to express the fusion protein with 100 μM isopropyl β-D-thiogalactopyranoside (IPTG). The recombinant protein was bound to a glutathione-agarose column (Sigma) and cleaved with thrombin (Pharmacia; Piscataway, N.J.). The purified protein was dialyzed against PBS at 4° C. and stored at −70^{c}C. The purity and integrity of the protein were determined by size separation using SDS-polyaclrylamide gel electrophoresis (PAGE), Coomassie blue staining, and Western blotting with the anti-human moesin mAb clone 38/87. The protein was quantitated by densitometric analysis of recombinant moesin and known amounts of BSA, which were used to construct a standard curve.

Analysis of Cytokine Secretion and Expression

Epithelial cells were grown as confluent monolayer's in 24-well tissue culture plates. After the cells reached confluence, the culture medium was changed and the cells were incubated with the disaccharides, with the addition of TNF-α. Disaccharides were added to the cells 1 hour before adding TNF-α. The disaccharides and TNF-α (200 ng/ml) were incubated with the cells for 24 hours. Following culture, the supernatants were harvested and analyzed for cytokine secretion. Each experiment was performed in duplicate.

In experiments in which the effect of anti-moesin antibodies was tested, anti-moesin and control antibodies were added at a concentration of 1.2 μg/ml and incubated for 30 minutes at 37° C. The cells were then washed, after which the disaccharide (1 ng/ml) was added for an additional 30 minutes at 37° C. Subsequently, TNF-α (200 ng/ml) was added and the cells were incubated for 18 hours at 37° C. Following culture, the supernatant was collected and assayed for IL-8 and IL-1β concentrations.

Cytokine ELI SA

IL-8 concentration was measured by ELISA. Briefly. 96-well plates were coated with polyclonal goat anti-human IL-8 antibodies (R&D Systems: Minneapolis, Minn.), as capturing antibodies. Following incubation with the tested supernatants at 37° C., for 1 hour, and washing three times, polyclonal rabbit anti-human antibodies (Endogen. Boston, Mass.) were added as detecting antibodies. Alkaline phosphatase-conjugated mouse anti-rabbit IgG Ab (Sigma) was used as a second-step antibody. The concentrations of the mouse anti-rabbit and rabbit anti-human antibodies were standard concentrations. Both were incubated at 37° C. for 1 hour, followed by three washings. The bound antibodies were visualized by using the alkaline phosphatase substrate p-nitrophenylphosphate (Sigma). IL-1β concentration was measured by an ELISA kit (Genzyme, Cambridge, Mass.) according to the manufacturer's instructions.

Heparin-Disaccharide Binds to Moesin

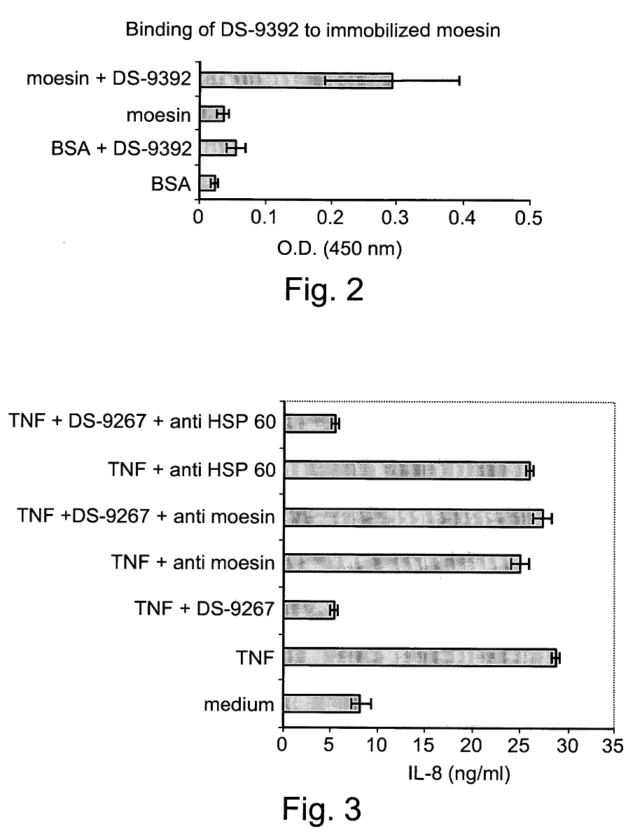

Since moesin binds to heparin and heparan sulfate (1), disaccharide (DS) derived from heparin was also tested to see if it could also bind to moesin. FIG. 2 shows that Heparin-DS binds to moesin, and not to BSA, as detected by antibody to heparan sulfate which recognized the DS.

Soluble Moesin and Anti Moesin Antibody Inhibit DS Activity on HT-29 Cells

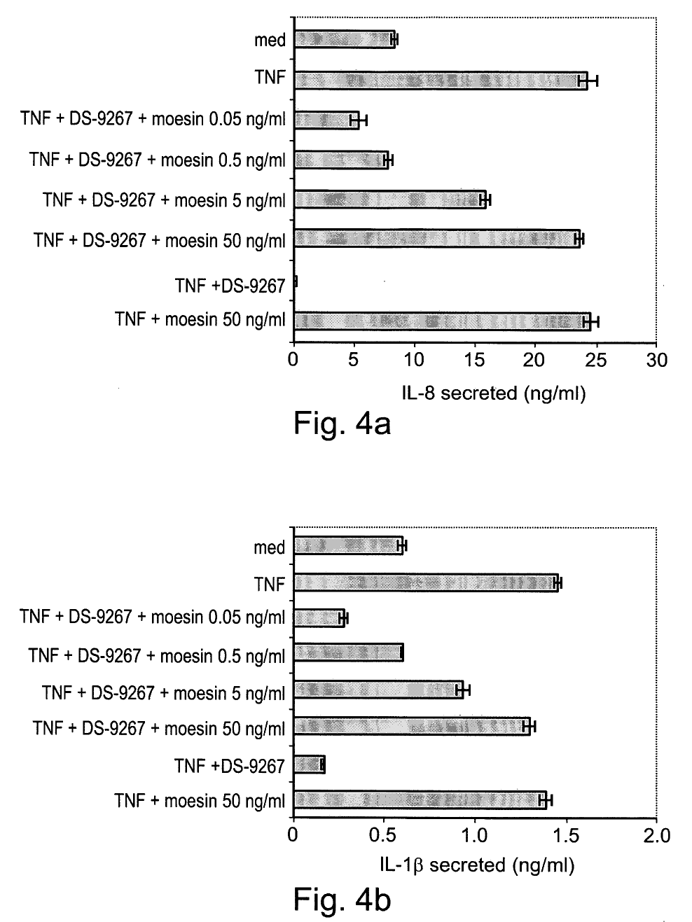

It has been shown that disaccharide molecules derived from heparin and from heparan sulfate can inhibit the secretion of IL-8 and IL-1β by HT-29 cells. Moreover, the DS molecules show a dose-dependent inhibition of both spontaneous and TNFα-stimulated cytokine secretion (5). Since DS binds to moesin, which is expressed on the surface of HT-29 cells, blocking moesin by anti-moesin specific antibodies was examined to determine whether it would inhibit the activation induced by DS on these cells. The cells were incubated with anti-moesin antibody (or control antibody), after which DS was added to the culture. Subsequently, the cells were treated with TNFα and the secretion of IL-8 was assessed. As shown in FIG. 3, the anti-moesin antibody specifically antagonized the inhibitory effect of the DS. To further verify that moesin bound to the DS, HT-29 cells were treated with the DS that was pre-incubated with increasing concentrations of recombinant human moesin and stimulated by TNFα. As shown in FIG. 4, the recombinant moesin antagonized the inhibitory effect of the DS in a dose-dependent manner. These results suggested that similar to the membrane associated moesin, the recombinant moesin bound the DS and thereby competed its effect on the cells. Taken together, the competition and blocking experiments indicate that the DS was acting via interaction with cell-surface moesin.

Soluble Moesin and Anti-Moesin Antibody Inhibit DS Activity on T Cells.

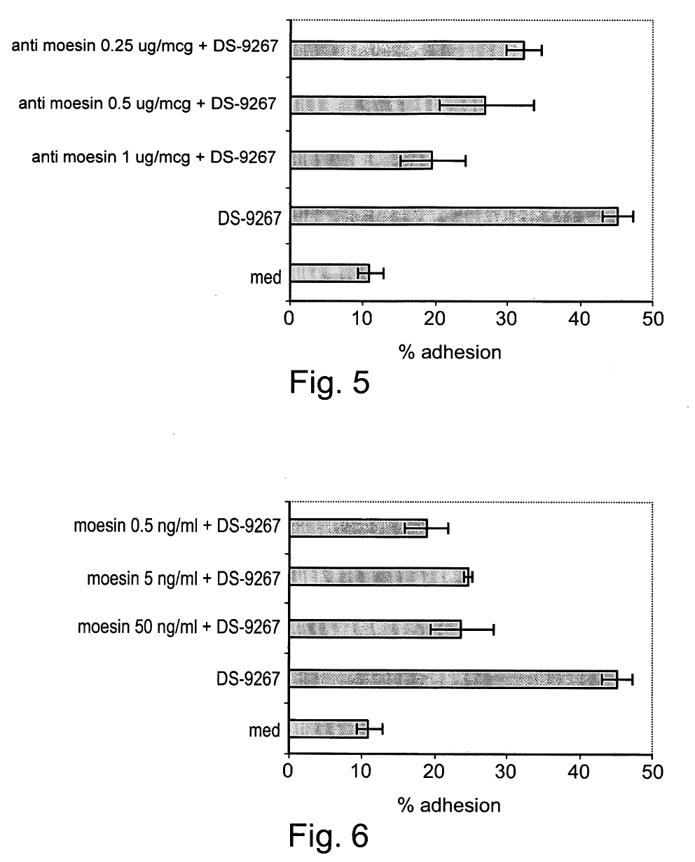

It was shown that certain heparin- and heparan sulfate-derived DS induced, in a dose-dependent manner, the adhesion of human T cells to both ECM and immobilized fibronectin (6). This adhesion appears to involve β1 integrin recognition and activation and is associated with specific intracellular activation pathways (6). Since moesin is expressed on T cells, antibody to moesin was examined for the potential to block the ability of DS to induce adhesion in T cells. FIG. 5 shows that indeed moesin antibody inhibited the adhesion of T cells to fibronectin induced by DS. Furthermore. as FIG. 6 shows, soluble moesin bound to the DS and thereby inhibited its induced adhesion of the T cells. This is another experimental system, which suggests that DS mediated its activity through binding to moesin.

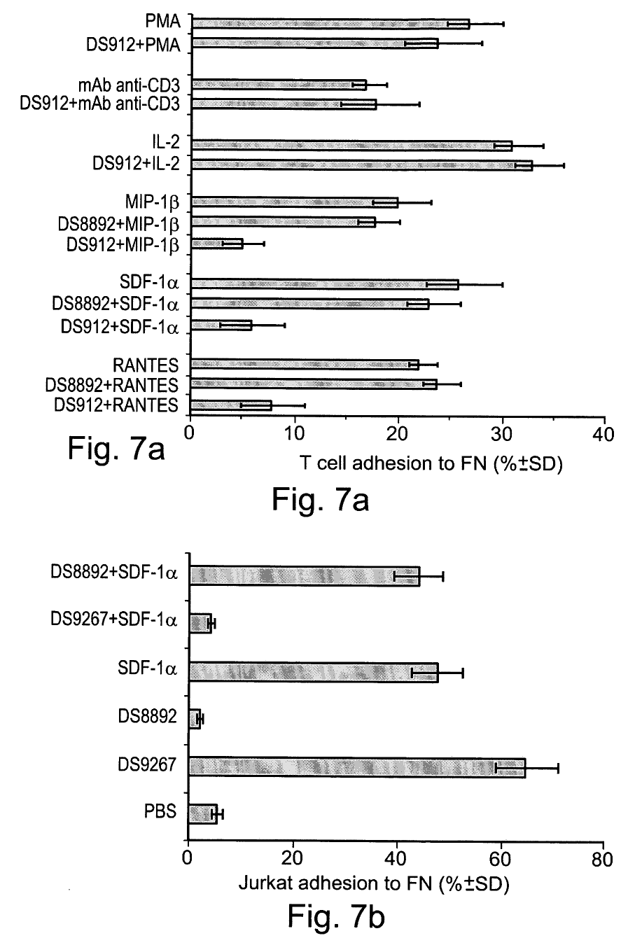

FIG. 7 shows that exposure of T-cells to these DS also showed that subsequent exposure of these T-cells to pro-adhesive chemokines, such as MIP-1β or RANTES, but not to other pro-adhesive stimuli, such as interleukin-2 or CD3 cross-linking, resulted in inhibition of T-cell adhesion and migration through FN. Without wishing to be limited by a single hypothesis, binding of the DS to moesin would appear to promote inhibition of T-cell adhesion and migration.

### Example 2

### Methods and Compositions for Administration

The saccharides of the present invention, and their homologues, derivatives or related compounds, hereinafter referred to as the “‘therapeutic agents of the present invention”, can be administered to a subject by various ways, which are well known in the art. Hereinafter the term “therapeutic agent” includes any saccharide-like material, or any material having a saccharide-like activity with regard to moesin, wherein saccharide activity with regard to moesin is described above.

The term “subject’” refers to the human or lower animal to which the therapeutic agent is administered. For example, administration may be done topically (including ophthalmically, vaginally, rectally, intranasally or by inhalation), orally, or parenterally, for example by intravenous drip or intraperitoneal, subcutaneous, or intramuscular injection.

Formulations for topical administration may be included but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, capsules or tablets. Thickeners, diluents, flavorings, dispersing aids, emulsifiers or binders may be desirable.

Formulations for parenteral administration may include but are not limited to sterile aqueous solutions which may also contain buffers, diluents and other suitable additives.

Dosing is dependent on the severity of the symptoms and on the responsiveness of the subject to the therapeutic agent. Persons of ordinary skill in the art can easily determine optimum dosages, dosing methodologies and repetition rates.

In one embodiment, the dose of a compound of the invention administered ranges from about 0.1 mg to about 1000 mg. In another embodiment, the dose administered ranges from about 1 mg to about 100 mg. In a further embodiment, the dose administered ranges from about 5 mg to about 50 mg. In yet another embodiment, the dose administered ranges from about 10 mg to about 30 mg.

In another embodiment, the dose of administration ranges from about 1 ng/kg of body weight to about 10 gr/kg of body weight. In a more preferred embodiment, the range is about 10 ng/kg of body weight to about 5 gr/kg of body weight. In another embodiment, the range is about 0.05 mg/kg of body weight to about 50 mg/kg of body weight. In a further embodiment, the dose administered ranges from about 0.1 mg/kg of body weight to about 10 mg/kg of body weight. In an additional embodiment, the dose administered ranges from about 0.1 mg/kg of body weight to about 1.0 mg/kg of body weight. In another embodiment, the dose administered is about 0.3 mg/kg of body weight.

In one embodiment, the dose is administered at a frequency of about once every 30 days to about once every day. In another embodiment, the dose is administered at a frequency of about once every 7 days to about once every day. In a further embodiment, the dose is administered at a frequency of about once every day.

### Example 3

### Methods and Indications of Treatment Using the Compounds

As noted above, the therapeutic agents of the present invention are believed to be effective inhibitors of inflammatory reaction, as well as for diseases with an inflammatory component. The following example is an illustration only of a method of treating an inflammatory condition and any other suitable condition with the therapeutic agent of the present invention, and is not intended to be limiting.

The method includes the step of administering a therapeutic agent, in a pharmaceutically acceptable carrier, to a subject to be treated. The therapeutic agent is administered according to an effective dosing methodology, preferably until a predefined endpoint is reached, such as the absence of a symptom of the inflammatory condition and any other suitable condition in the subject, or the prevention of the appearance of such a condition or symptom in the subject.

The present invention also discloses methods for treating malignancies. Hereinafter, the term “treatment” includes both the prevention of the genesis of the malignancy, as well as the substantial reduction or elimination of malignant cells or symptoms associated with the development and metastasis of malignancies. Malignancies for which the therapeutic agents of the present invention are useful include all metastatic tumors. Examples of tumors for which such a treatment would be effective include, but are not limited to, breast cancers such as infiltrating duct carcinoma of the breast or other metastatic breast cancers, lung cancers such as small cell lung carcinoma, bone cancers, bladder cancers such as bladder carcinoma, rhabdomyosarcoma, angiosarcoma. adenocarcinoma of the colon, prostate or pancreas, or other metastatic prostate or colon cancers, squamous cell carcinoma of the cervix, ovarian cancer, malignant fibrous histiocytoma, skin cancers such as malignant melanoma, lymphomas, leukemia, leiomyosarcoma, astrocytoma, glioma and heptocellular carcinoma. Such treatment may optionally and preferably be performed by systemic administration of the therapeutic agent according to the present invention. A preferred route of administration is oral. Alternative routes of administration include, but are not limited to, intranasal, intraocular, sub-cutaneous and parenteral administration. Such treatment may be performed topically, for example for skin malignancies, including but not limited to, metastatic melanoma. Other routes of administration and suitable pharmaceutical formulations thereof are also possible as previously described.

The compounds of the present invention can be used to treat a variety of conditions, including, but not limited to, those listed is U.S. Pat. No. 5,861,382.

More particularly, the compounds according to the present invention can also be used to treat central nervous system neurodegenerative disorders such as, but not limited to, Parkinson's, Alzheimer's, Kuru and Creutzfeldt-Jakob's diseases, basal ganglia degenerative diseases, motorneuron diseases, Scrapie, Mad cow disease, spongyform encephalopathy, Subacute Sclerosing Pan-Encephalitis (SSPE) and peripheral tissue disorders such as, but not limited to, acute respiratory distress syndrome, amyotrophic lateral sclerosis, atheroscierotic cardiovascular disease and multiple organ dysfunction, all of which were previously shown to be associated with formation and/or overproduction of oxidants.

The compounds of the present invention can also inhibit the replication or infectivity of a virus or a virus-infected cell. This can be shown in vitro using a variety of assays known in the art, or described herein. In certain embodiments, such assays may use cells of a cell line, or cells from a patient. In specific embodiments, the cells may be infected with a virus prior to the assay, or during the assay. The cells may be contacted with a virus. In certain other embodiments, the assays may employ cell-free viral cultures.

In one embodiment, a compound of the present invention can be shown to treat or prevent a viral disease by contacting cultured cells that exhibit an indicator of a viral reaction (e.g., formation of inclusion bodies) in vitro with the compound, and comparing the level of the indicator in the cells contacted with the compound with the level of the indicator in cells not so contacted, wherein a lower level in the contacted cells indicates that the compound has activity in treating or preventing viral disease. Cell models that can be used for such assays include, but are not limited to, viral infection of T lymphocytes (Selin et al., 1996, J. Exp. Med. 183:2489-2499); hepatitis B infection of dedifferentiated hepatoma cells (Raney et al., 1997, J. Virol. 71:1058-1071); viral infection of cultured salivary gland epithelial cells (Clark et al., 1994, Autoimmunity 18:7-14); synchronous HIV-1 infection of CD4 sup.+ lymphocytic cell lines (Wainberg et al., 1997, Virology 233:364-373); viral infection of respiratory epithelial cells (Stark et al., 1996, Human Gene Ther. 7:1669-1681); and amphotrophic retroviral infection of NIH-3T3 cells (Morgan et al., 1995, J. Virol. 69:6994-7000).

In another embodiment, a compound of the invention can be demonstrated to have activity in treating or preventing viral disease by administering the compound to a test animal having symptoms of a viral infection, such as characteristic respiratory symptoms in animal models, or which test animal does not exhibit a viral reaction and is subsequently challenged with an agent that elicits a viral reaction, and measuring the change in the viral reaction after the administration of the compound, wherein a reduction in the viral reaction or a prevention of the viral reaction indicates that the compound has activity in treating or preventing viral disease. Animal models that can be used for such assays include, but are not limited to, guinea pigs for respiratory viral infections (Kudlacz and Knippenberg, 1995, Inflamm. Res. 44:105-110); mice for influenza virus infection (Dobbs et al., 1996, J. Immunol. 157:1870-1877); lambs for respiratory syncitial virus infection (Masot et al., 1996, Zentralbl. Veterinarmed. 43:233-243); mice for neurotrophic virus infection (Barna et al., 1996, Virology 223:331-343); hamsters for measles infection (Fukuda et al., 1994, Acta Otolaryngol. Suppl (Stockh.) 514:111-116); mice for encephalomyocarditis infection (Hirasawa et al., 1997, J. Virol. 71:4024-4031); and mice for cytomegalovirus infection (Orange and Biron, 1996, J. Immunol. 156:1138-1142). In certain embodiments of the invention more than one compound of the invention is administered to a test animal, virus, or viral-infected cell.

Viruses and viral infections that can be treated or prevented by administering a compound of the invention include, but are not limited to, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, Morbilliviruses such as the paramixoviruses family, such as measles virus, Rinderpest virus and Canine Distemper virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

Moreover, the compounds of the invention can be used to treat or prevent a parasitic infection or disease. Examples of such parasitic infection or disease include, but are not limited to, protozoan infections or diseases such as amebiasis, babesiosis, Chagas' disease, leishmaniasis, toxoplasmosis, malaria, giardiasis and pneumocystosis; and helminthes infections or diseases such as cysticercosis, echinococcosis, paragonimiasis, toxocariasis, trichnosis, ascariasis, clonorchiasis, dracunculiasis, filariasis, schistosomiasis and strongyloidiasis.

In addition, the compounds of the invention can be used to treat or prevent a bacterial infection or disease. Examples of such bacterial infection or disease include, but are not limited to those caused by micrococcus, staphylococcus, streptococcus, lactococcus, enterococcus, leuconostoc, pediococcus, aerococcus, lactobacillus, kurthia, arthrobacter, clostridium, bacillus, alcaligenes, pseudomonas, klebsiella, shigella, salmonella, escherichia, other enteric genera, aeromonas, chromobacterium and neisseria.

In addition, the compounds of the present invention are useful in the treatment of the disorders listed in WO-A-98/05635. For ease of reference, part of that list is now provided: inflammation or inflammatory diseases, dermatological disorders, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; angiogenesis, malignant pleural effusion; cerebral ischaemia, ischaemic heart-disease, osteoarthritis, rheumatoid arthritis, osteoporosis, asthma, multiple sclerosis, neurodegeneration, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis. In addition, the compounds of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumor immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilizing specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behavior; as analgesics; treating specific deficiency disorders; in treatment of e.g. psoriasis, in human or veterinary medicine.

Moreover, the compounds of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extra cellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitro-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumor cerebri; Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery; bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

As used herein, the term “cancer” refers to various types of malignant neoplasms, most of which can invade surrounding tissues, and may metastasize to different sites, as defined by Stedman's medical Dictionary 25th edition (Hensyl ed., 1990). Examples of cancers which may be treated by the compounds of the present invention include, but are not limited to, brain, ovarian, colon, prostate, kidney, bladder, breast, lung, oral and skin cancers which exhibit inappropriate PTK activity. These cancers can be further broken down. For example, brain cancers include glioblastoma multiforme, anaplastic astrocytoma, astrocytoma, ependyoma, oligodendroglioma, medulloblastoma, meningioma, sarcoma, hemangioblastoma, and pineal parenchymal. Likewise, skin cancers include melanoma and Kaposi's sarcoma. PTKs have been associated with the development of cancer. Some of the above mentioned PTK receptors, like EGFR and PDGFR, are over-expressed in many tumors and/or are persistently activated by autocrine loops have been demonstrated. Specifically, PDGFR has been associated with glioblastoma, melanoma and lung, ovarian, and prostate cancer.

Herein, the term “treating” includes abrogating, substantially inhibiting, slowing or reversing the progression of a disease, substantially ameliorating clinical symptoms of a disease or substantially preventing the appearance of clinical symptoms of a disease.

Herein, the term “preventing” refers to a method for barring an organism from acquiring a disorder or disease in the first place.

The term “organism” refers to any living entity comprised of at least one cell. A living organism can be as simple as, for example, a single eukaryotic cell or as complex as a mammal, including a human being.

The term “therapeutically effective amount” refers to that amount of the compound being administered which will relieve to some extent one or more of the symptoms of the disorder being treated.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of improving, preventing or treating a condition selected from the group consisting of parasitic infection, bacterial infection, viral infection, nerve injury or damage, nerve regeneration, Downs syndrome, inflammatory disease, brain injury, lung cancer, cancer, head and neck cancer, skin cancer, pancreatic cancer, metastatic cancer, GI cancer, GI disease, skin disease, allergy and autoimmune disease, wherein said method comprises administering a compound of the formula: 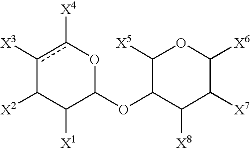
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

2. The method of claim 1, wherein: X₁ is selected from the group consisting of —OH, —OSO₃H, —OSO₃⁻, —NHSO₃H and —NHSO₃⁻; X₂ is —OH; X₃ is selected from the group consisting of —OH and hydrogen; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H and CO₂H; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

3. The method of claim 1, wherein said compound has the formula: 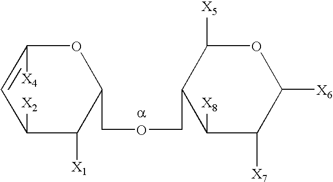
wherein:
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

4. The method of claim 3, wherein: X₁ is selected from the group consisting of —OH, —OSO₃H, —OSO₃⁻, —NHSO₃H and —NHSO₃⁻; X₂ is —OH; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H, —CH₂OSO₃⁻, —C(O)O⁻ and —C(O)OH; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

5. The method of claim 3, wherein: X₁ is —OSO₃⁻; X₂ is —OH; X₄ is —C(O)O⁻; Xs is —CH₂OSO₃⁻; X₆ is —OH; X₇ is —NHSO₃⁻; and X₈ is —OH.

6. The method of claim 3, wherein: X₁ is —OSO₃⁻; X₂ is —OH; X₄ is —C(O)O⁻; X₅ is —CH₂OH; X₆ is —OH; X₇ is —NHSO₃⁻; and X₈ is —OH.

7. The method of claim 3, wherein said condition is selected from the group consisting of measles infection, rabies infection, adenovirus infection, parasitic infection, shigella infection, pseudomonas infection, helicobacter infection, streptococcus infection, and neisseria infection.

8. The method of claim 3, wherein said condition is selected from the group consisting of nerve injury or damage, central nervous system (CNS) inflammatory disease, brain injury, lung cancer, CNS cancer, head and neck cancer, skin cancer, pancreatic cancer, metastatic cancer and skin disease.

9. A method for inhibiting chemokine-dependent migration or chemokine-dependent adhesion of cells expressing moesin, comprising mediating the inhibition of the chemokine-dependent activity through at least one activation or reduction of moesin activity or at least one modification of existing moesin activity.

10. The method of claim 1, wherein said cells comprise immune, immune-related, tumor or malignant cells.

11. The method of claim 9, wherein said activation or modification of moesin activity comprises an activation or modification that can be mediated through binding of a saccharide to meosin.

12. The method of claim 11, wherein said saccharide is sulfated.

13. The method of claim 11, wherein said saccharide is a disaccharide.

14. The method of claim 13, wherein said disaccharide is sulfated.

15. The method of any of claim 9, further comprising administering a disaccharide or a derivative thereof to a subject.

16. The method of claim 15, wherein said disaccharide or derivative thereof has the formula: 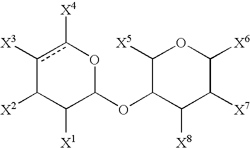
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

17. The method of claim 16, wherein: X₁ is selected from the group consisting of —OH, —OSO₃H, —OSO₃⁻, —NHSO₃H and —NHSO₃⁻; X₂ is —OH; X₃ is selected from the group consisting of —OH and hydrogen; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H and CO₂H; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

18. The method of claim 16, wherein said disaccharide or derivative thereof has the formula: 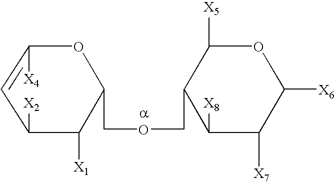
wherein:
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

19. The method of claim 18, wherein: X₁ is selected from the group consisting of —OH, —OSO₃H, —OSO₃⁻, —NHSO₃H and —NHSO₃⁻; X₂ is —OH; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H, —CH₂OSO₃⁻, —C(O)O⁻ and —C(O)OH; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

20. The method of claim 18, wherein: X₁ is —OSO₃⁻; X₂ is —OH; X₄ is —C(O)O⁻; X₅ is —CH₂OSO₃⁻; X₆ is —OH; X₇ is —NHSO₃⁻; and X₈ is —OH.

21. The method of claim 18, wherein: X₁ is —OSO₃⁻; X₂ is —OH; X₄ is —C(O)O⁻; X₅ is —CH₂OH; X₆ is —OH; X₇ is —NHSO₃⁻; and X₈ is —OH.

22. A method for modulating moesin-mediated intracellular signaling, wherein said signaling is capable of being mediated through an effect of a saccharide binding to moesin, comprising altering moesin activity in cells such that the moesin-mediated intracellular signaling is modulated.

23. The method of claim 22, wherein said moesin activity is altered through administration of a saccharide or derivative thereof.

24. The method of claim 23, wherein the saccharide or derivative thereof is derived from heparin or heparan sulfate.

25. The method of claim 23, wherein the saccharide or derivative thereof is sulfated.

26. The method of claim 23, wherein the saccharide or derivative thereof is a disaccharide.

27. The method of claim 23, wherein said disaccharide or derivative thereof has the formula: 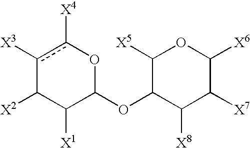
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

28. The method of claim 27, wherein: X₁ is selected from the group consisting of —OH, —OSO₃H, —OSO₃⁻, —NHSO₃H and —NHSO₃⁻; X₂ is —OH; X₃ is selected from the group consisting of —OH and hydrogen; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H and CO₂H; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

29. The method of claim 27, wherein said disaccharide or derivative thereof has the formula: 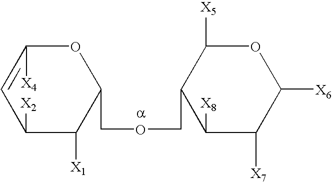
wherein:
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

30. The method of claim 29, wherein: X₁ is selected from the group consisting of —OH, —OSO₃H, —OSO₃⁻, —NHSO₃H and —NHSO₃⁻; X₂ is —OH; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H, —CH₂OSO₃⁻, —C(O)O⁻ and —C(O)OH; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

31. The method of claim 29, wherein: X₁ is —OSO₃⁻; X₂ is —OH; X₄ is —C(O)O⁻; X₅ is —CH₂OSO₃⁻; X₆ is —OH; X₇ is —NHSO₃⁻; and X₈ is —OH.

32. The method of claim 29, wherein: X₁ is —OSO₃⁻; X₂ is —OH; X₄ is —C(O)O⁻; X₅ is —CH₂OH; X₆ is —OH; X₇ is —NHSO₃⁻; and X₈ is —OH.

33. A method for modifying at least one effect of at least one external influence on an eukaryotic cell, wherein the at least one effect is affected by binding of a saccharide to moesin, comprising modification by the saccharide of moesin, thereby modifying the effect.

34. The method of claim 33, wherein the effect is increased.

35. The method of claim 33, wherein the effect is decreased.

36. A method for modifying at least one effect of at least one external influence on an eukaryotic cell, wherein the at least one effect is mediated by binding of a saccharide to moesin, comprising altering the at least one effect by binding a substance to meosin, thereby modifying the effect.

37. The method of claim 36, wherein the saccharide or derivative thereof is derived from heparin or heparan sulfate.

38. The method of claim 36, wherein the saccharide or derivative thereof is sulfated.

39. The method of claim 36, wherein the saccharide or derivative thereof is a disaccharide.

40. The method of claim 36, wherein said disaccharide or derivative thereof has the formula: 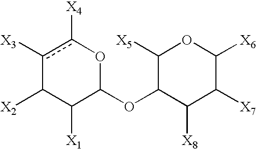
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

41. The method of claim 40, wherein: X₁ is selected from the group consisting of —OH, —OSO₃H, —OSO₃⁻, —NHSO₃H and —NHSO₃⁻; X₂ is —OH; X₃ is selected from the group consisting of —OH and hydrogen; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H and CO₂H; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

42. The method of claim 40, wherein said disaccharide or derivative thereof has the formula: 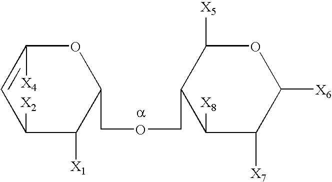
wherein:
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy.

43. The method of claim 42, wherein: X₁ is selected from the group consisting of —OH, —OSO₃H, —OSO₃⁻, —NHSO₃H and —NHSO₃⁻; X₂ is —OH; X₄ is selected from the group consisting of —CH₂OSO₃H, —CH₂OSO₃⁻, —C(O)O⁻, —C(O)OH and hydrogen; X₅ is selected from the group consisting of —CH₂OH, —CH₂OSO₃H, —CH₂OSO₃⁻, —C(O)O⁻ and —C(O)OH; X₆ is —OH; X₇ is selected from the group consisting of —OSO₃H, —OSO₃⁻, —NHSO₃H, —NHSO₃⁻, —NHC(O)CH₃, —NH₂ and —NH₃⁺; and X₈ is —OH.

44. The method of claim 42, wherein: X₁ is —OSO₃⁻; X₂ is —OH; X₄ is —C(O)O⁻; X₅ is —CH₂OSO₃⁻; X₆ is —OH; X₇ is —NHSO₃⁻; and X₈ is —OH.

45. The method of claim 42, wherein: X₁ is —OSO₃⁻; X₂ is —OH; X₄ is —C(O)O⁻; X₅ is —CH₂OH; X₆ is —OH; X₇ is —NHSO₃⁻; and X₈ is —OH.

46. A method for blocking cell migration or adhesion, comprising administering a modulating agent capable of mimicking binding of a saccharide to moesin, wherein the cell migration or adhesion is capable of being blocked by a saccharide binding to said moesin.

47. The method of claim 46, wherein said modulating agent is administered to treat a disease that is mediated by cell migration or adhesion.

48. The method of claim 46, wherein said modulating agent is administered to treat a disease characterized by malignant cell growth.

49. A method for blocking cytokine secretion, comprising administering a modifying agent for modifying moesin activity through a mechanism activated by saccharide binding to moesin.

50. The method of claim 49, wherein said modifying agent is used to treat a disease mediated through a cytokine.

51. Use of a compound of the formula: 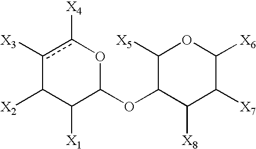
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy,
wherein said use is for treating a condition selected from the group consisting of parasitic infection, bacterial infection, viral infection, nerve injury or damage, nerve regeneration, Downs syndrome, inflammatory disease, brain injury, lung cancer, cancer, head and neck cancer, skin cancer, pancreatic cancer, metastatic cancer, GI cancer, GI disease, skin disease, allergy and autoimmune disease.

52. Use of a compound of the formula: 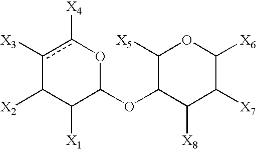
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy,
wherein said use is for inhibiting chemokine-dependent migration or chemokine-dependent adhesion of cells expressing moesin by mediating the inhibition of the chemokine-dependent activity through at least one activation of moesin or at least one modification of existing moesin activity.

53. Use of a compound of the formula: 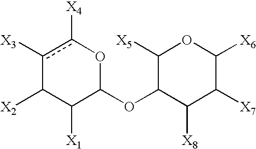
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy,
wherein said use is for modulating moesin-mediated intracellular signaling, wherein said signaling is capable of being mediated through an effect of a saccharide binding to moesin by altering moesin activity in cells such that the moesin-mediated intracellular signaling is modulated.

54. Use of a compound of the formula: 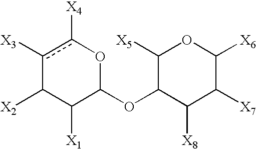
wherein:
the dotted line is an optional double bond;
X₁ is selected from the group consisting of hydroxyl, C₁ to C₁₂ alkoxy, C₁ to C₁₂ substituted alkoxy, sulfate, amino, (monosubstituted) amino and (disubstituted)amino;
X₂ is selected from the group consisting of hydroxyl, C₁ to C₁₂ alkoxy and C₁ to C₁₂ substituted alkoxy;
X₃ is selected from the group consisting of hydrogen, hydroxyl, C₁ to C₁₂ alkoxy and C₁ to C₁₂ substituted alkoxy;
X₄ is selected from the group consisting of C₁ to C₁₂ alkyl, C₁ to C₁₂ substituted alkyl, hydrogen and the formula —C(O)OR, wherein R is absent or selected from the group consisting of C₁ to C₁₂ alkyl, C₁ to C₁₂ substituted alkyl and hydrogen;
X₅ is selected from the group consisting of C₁ to C₁₂ alkyl, C₁ to C₁₂ substituted alkyl, C₁ to C₁₂ alkoxycarbonyl and C₁ to C₁₂ substituted alkoxycarbonyl;
X₆ is selected from the group consisting of hydroxyl, C₁ to C₁₂ alkoxy and C₁ to C₁₂ substituted alkoxy;
X₇ is selected from the group consisting of hydroxyl, C₁ to C₁₂ alkoxy, C₁ to C₁₂ substituted alkoxy, sulfate, amino, (monosubstituted) amino and (disubstituted)amino; and
X₈ is selected from the group consisting of hydroxyl, C₁ to C₁₂ alkoxy and C₁ to C₁₂ substituted alkoxy,
wherein said use is for modifying at least one effect of at least one external influence on an eukaryotic cell, wherein the at least one effect is mediated by binding of a saccharide to moesin, by, modification by the saccharide of moesin, thereby modifying the effect.
